(19) United States
(12) Patent Application Publication
(10) Pub. No.: US 2024/0417439 A1
(43) Pub. Date: Dec. 19, 2024

(54) RECOMBINANT POLYPEPTIDE FOR DISRUPTING INTERACTION OF EAG2 AND KVß2 AND THERAPEUTIC APPLICATIONS THEREOF IN CANCER TREATMENT

(71) Applicant: The Hospital for Sick Children, Toronto (CA)

(72) Inventors: Xi HUANG, Markham (CA); Weifan DONG, Toronto (CA)

(21) Appl. No.: 18/703,101

(22) PCT Filed: Oct. 21, 2022
(86) PCT No.: PCT/CA2022/051560

§ 371 Date: Apr. 19, 2024

Related U.S. Application Data
(60) Provisional application No. 63/270,858, filed on Oct. 22, 2021.

(51) Int. Cl. C07K 14/705; A61K 38/00; A61P 35/00; G01N 33/50

(52) U.S. Cl. CPC C07K 14/705; A61P 35/00; G01N 33/5011; A61K 38/00; C07K 2319/10; G01N 2500/02

(57) ABSTRACT

Herein in provided a recombinant polypeptide comprising a polypeptide for preventing or reducing interaction between the human proteins EAG2 and KvB2, and a cell-penetrating peptide. The polypeptide may comprise a portion of Kvß2 from or encompassing a region that is important for the interaction between the two proteins, such as, e.g., a contiguous portion of human Kvß2 (SEQ ID NO: 26) encompassing at least amino acids 90 to 114 thereof. Retro-inverso polypeptides based on such recombinant polypeptides are also described. The EAG2-Kvß2 complex is herein identified as a therapeutic target for certain cancers. Herein are also disclosed therapeutic applications of the recombinant polypeptide in treatment of such cancers, including gliomas, such as low-grade gliomas or glioblastomas, which may be recurrent and/or resistant to conventional treatment.

### CROSS-REFERENCE TO RELATED APPLICATIONS

This application claims priority from U.S. Provisional Application No. 63/270,858 filed Oct. 22, 2021 and entitled “RECOMBINANT POLYPEPTIDE FOR DISRUPTING INTERACTION OF EAG2 AND KVβ2 AND THERAPEUTIC APPLICATIONS THEREOF IN CANCER TREATMENT”, the contents of which are incorporated by reference.

### FIELD

The present disclosure relates generally to polypeptides for disrupting protein-protein interactions. More particularly, the present disclosure relates to a recombinant polypeptide for preventing or reducing interaction between EAG2 and Kvß2.

### BACKGROUND

Glioblastoma (GBM) is the most common and aggressive primary brain cancer. Standard-of-care includes surgery, radiation, and chemotherapy using the DNA alkylating agent temozolomide (TMZ), and leaves patients with median survival of about 15 months. As TMZ induces DNA damage to promote GBM cell death, it causes profound side effects in non-tumoral cells, leading to neural, gastrointestinal, and hematologic toxicity. Furthermore, TMZ alters the tumor cell genome, which promotes the emergence of therapy-resistant clones and eventual treatment failure. Since U.S. Food and Drug Administration (FDA) approval of TMZ for treatment of refractory anaplastic astrocytoma in 1999 and newly diagnosed GBM in 2005¹, no clinical trials for other molecular targets have advanced into the clinics as approved drugs. These points highlight the need to identify non-conventional molecular targets in GBM and related cancers.

It is, therefore, desirable to provide additional therapeutic targets and therapeutics for GBM and related cancers.

### SUMMARY

It is an object of the present disclosure to obviate or mitigate at least one disadvantage of previous.

In one aspect, there is provided a recombinant polypeptide comprising a) a polypeptide for preventing or reducing interaction between EAG2 and Kvß2 comprising i) at least 5 contiguous amino acids from a region of human Kvß2 selected from the group consisting of amino acids 1 to 67 thereof, amino acids 90 to 114 thereof, and amino acids 343 to 355 thereof, or ii) a polypeptide that is at least 70% identical to i); and b) a cell-penetrating peptide.

In one aspect, there is provided a recombinant polypeptide comprising: a) a polypeptide for preventing or reducing interaction between EAG2 and Kvß2 comprising: i) a contiguous portion of human Kvß2 encompassing at least amino acids 90 to 114 thereof, or ii) a polypeptide that is at least 70% identical to i); and b) a cell-penetrating peptide.

In one aspect, there is provided a retro-inverso polypeptide based on any one of the recombinant polypeptides described herein.

In one aspect, there is provided a nucleic acid encoding the recombinant polypeptide as herein described.

In one aspect, there is provided a vector comprising the nucleic acid as herein described.

In one aspect, there is a provided a host cell comprising the nucleic acid as herein described or the vector as herein described.

In one aspect, there is provided a composition comprising the recombinant polypeptide as herein described, the nucleic acid as herein described, or the vector as herein described; together with an excipient, diluent, or carrier.

In one aspect, there is provided a pharmaceutical composition comprising a recombinant polypeptide as herein described, the nucleic acid as herein described, or the vector as herein described; together with a pharmaceutically acceptable excipient, diluent, or carrier.

In one aspect, there is provided a method of preventing or reducing interaction of EAG2 and Kvß2 in a cell comprising: contacting the cell with the recombinant polypeptide as herein described, the nucleic acid as herein described, the vector as herein described, or the pharmaceutical composition as described herein.

In one aspect, there is provided a use of the recombinant polypeptide as herein described, the nucleic acid as herein described, the vector as herein described, or the pharmaceutical composition as described herein for preventing or reducing interaction of EAG2 and Kvß2 in a cell.

In one aspect, there is provided a use of the recombinant polypeptide as herein described, the nucleic acid as herein described, the vector as herein described, or the pharmaceutical composition as described herein for preparation of a medicament for preventing or reducing interaction of EAG2 and Kvß2 in a cell.

In one aspect, there is provided the recombinant polypeptide as herein described, the nucleic acid as herein described, the vector as herein described, or the pharmaceutical composition as described herein for use in preventing or reducing interaction of EAG2 and Kvß12 in a cell.

In one aspect, there is provided a method of treating cancer in a subject comprising: administering to the subject the recombinant polypeptide as herein described, the nucleic acid as herein described, the vector as herein described, or the pharmaceutical composition as described herein.

In one aspect, there is provided a use of the recombinant polypeptide as herein described, the nucleic acid as herein described, the vector as herein described, or the pharmaceutical composition as described herein for treatment of cancer in a subject.

In one aspect, there is provided a use of the recombinant polypeptide as herein described, the nucleic acid as herein described, the vector as herein described, or the pharmaceutical composition as described herein for preparation of a medicament for treatment of cancer in a subject.

In one aspect, there is provided the recombinant polypeptide as herein described, the nucleic acid as herein described, the vector as herein described, or the pharmaceutical composition as described herein for use in treatment of cancer in a subject.

In one aspect, there is provided a method of screening for a candidate therapeutic for cancer comprising: contacting a human cell with a test compound, wherein the human cell has an original level of interaction between EAG2 and Kvß2, measuring a level of interaction between EAG2 between Kvß2 after the step of contacting, and identifying the test compound as a candidate therapeutic for cancer if the measured level of the interaction between EAG2 and Kvß2 is reduced compared to the original level.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 depicts a series of images showing that tumor-specific expression of dominant negative eag or tumor-specific knockdown of Hk decreases tumor growth in a Drosophila melanogaster (fruit fly) model of GBM.

FIG. 2 depicts quantifications of tumor volume of Drosophila GBM with or without expression of dominant negative eag or tumor-specific knockdown of Hk

FIG. 3 depicts a series of images showing that dominant negative eag reduces tumor volume, the number of mitotic glia cells, and total glia cell number in Drosophila GBM.

FIG. 4 depicts quantifications of mitotic glia cell number of Drosophila GBM with or without expression of dominant negative eag.

FIG. 5 depicts a series of images showing that tumor-specific knockdown of Hk reduces tumor volume, the number of mitotic glia cells, and total glia cell number in Drosophila GBM.

FIG. 6 depicts quantifications of mitotic glia cell numbers of Drosophila GBM with or without tumor-specific knockdown of Hk.

FIG. 7 shows plots of overall survival probability (months) showing high EAG2 or Kvβ62 expression associates with worse survival of glioma patients.

FIG. 8 is a plot of EAG2 and Kvβ2 expression, showing that EAG2 and Kvβ62 are co-expressed in human gliomas (based on TCGA LGG-GBM dataset).

FIG. 9 shows that EAG2 is expressed in grade 2 astrocytoma, grade 3 astrocytoma, and GBM in human patients.

FIG. 10 shows that EAG2 and Kvβ2 proteins are expressed in human fetal neural stem cell (NPC) lines, GBM cell lines, and medulloblastoma (MB) cell lines.

FIG. 11 depicts quantifications of EAG2 and Kvβ2 protein expression levels in NPC, GBM, and MB cell lines.

FIG. 12 depicts a series of images showing that EAG2 or Kvβ2 knockdown decreases clonogenic growth of human GBM cells.

FIG. 13 shows that EAG2 or Kvβ2 knockdown decreases sphere forming ability of human GBM cells.

FIG. 14 depicts quantifications of sphere forming ability of GBM cells with or without EAG2 or Kvβ2 knockdown.

FIG. 15 shows that inducible EAG2 or Kvβ2 knockdown suppresses GBM growth and extends mouse survival. Combinatorial inducible EAG2 and Kvβ2 knockdown cooperatively suppress GBM growth.

FIG. 16 shows histological sections of GBM tumor sizes with or without EAG2 knockdown, Kvβ2 knockdown, or combinatorial EAG2 and Kvβ2 knockdown.

FIG. 17 depicts a series of images showing that EAG2 localizes at intracellular compartments at interphase, and it displays prominent plasma membrane localization during mitosis.

FIG. 18 shows that Kvß2 knockdown abrogates the mitosis-specific plasma membrane localization of EAG2.

FIG. 19 shows quantifications of the ratio of plasma membrane-localized EAG2 in total EAG2 with or without Kvß2 knockdown.

FIG. 20 shows that EAG2 or Kvß2 knockdown decreased the mitotic index and led to multinucleation (an indicator of aberrant mitosis) of GBM cells.

FIG. 21 shows that EAG2 localizes at synapse-like contact site between GBM cell process (GFP⁺) and neuronal process (Tuj1⁺). EAG2 localization juxtaposes pre-synaptic marker vGLUT1. EAG2 enriches at GBM-neuron contact sites that express post-synaptic marker PSD95, while EAG2 displays scattered localization in GBM cell not in contact with a neuron. Kvβ2 knockdown decreases EAG2 enrichment at GBM-neuron contact site. Quantifications show EAG2 level at GBM cell body, EAG2 level at tumor-microtubes, or GBM cell microtube length with or without Kvβ2 knockdown.

FIG. 22 shows that high EAG2 or Kvβ62 expression associates with synaptic gene expression at the leading edge or infiltrating region of human GBM (based on Ivy GBM Atlas).

FIG. 23 shows that EAG2 and Kvβ2 physically interact in GBM cell lines but not human fetal neural stem cell lines or mouse brains.

FIG. 24 shows that a series of Kvβ2 truncation mutants (f1-f9) have been generated.

FIG. 25 shows that Kvβ2 truncation mutants f1, f2, and f4 interact with EAG2, while f3, f5, or f6 does not. Co-IPs of overexpressed proteins are performed using HEK293T cells.

FIG. 26 shows that Kvβ2 truncation mutants f1, f2, and f4 interacts with EAG2.

FIG. 27 shows that f7, but not full length Kvβ2, interacts with EAG2.

FIG. 28 shows that f8, but not f9 or full length Kvβ2, interacts with EAG2, revealing amino acid 343-355 as an inhibitory sequence that prevents full length Kvß2 from interacting with EAG2.

FIG. 29 shows schematics of canonical full length Kvβ2, and Kvβ2 isoform 2, 3, 4, and 5.

FIG. 30 shows that Kvβ2 isoform 4, but not canonical full length Kvβ2, isoform 2, 3, or 5, physically interacts with EAG2.

FIG. 31 shows that GBM, but not non-tumor or non-GBM tumor cell lines, display high expression of Kvβ62 isoform 4.

FIG. 32 shows that monoclonal anti-Kvβ2 antibody (clone K17/70) recognizes full length Kvβ2 but not Kvβ2 isoform 4.

FIG. 33 shows that Kvβ2 isoform 4 confers full length Kvβ2 the ability to interact with EAG2.

FIG. 34 shows that Kvβ2 amino acid 79-158 (f4) contains two α-helices (K90-114 and K126-147). Designer interference peptides are generated by adding TAT cell-penetrating sequence and a linker to each α-helix.

FIG. 35 shows that designer interference peptide K90-114^{TAT} can be internalized by GBM cells.

FIG. 36 shows that 4-hour treatment using K90-114^{TAT}, but not K59-78^{TAT}, reduces physical interaction between endogenous EAG2 and Kvß2 in GBM cells. 8-hour or 16-hour treatment using K90-114^{TAT}, but not K59-78^{TAT}, decreases EAG2 protein level in GBM cells.

FIG. 37 show that K90-114^{TAT}, but not K59-78^{TAT}, decreases cell cycle ability of GBM cells.

FIG. 38 K90-114^{TAT}, but not other peptides (K59-78^{TAT}, K126-147^{TAT}, K344-355^{TAT}) decreases mitosis and increases apoptosis of GBM cells.

FIG. 39 shows that K90-114^{TAT}, but not other peptides (K59-78^{TAT}, K126-147^{TAT}K344-355^{TAT}), reduces the viability of multiple GBM cell lines (G411, G532, G508, G799, G800, G744r) at concentrations that do not impact human neural progenitor cells (hf6562, hf7450) or HEK293T cells.

FIG. 40 shows that K90-114^{TAT}, but not other peptides (K59-78^{TAT}, K126-147^{TAT}K344-355^{TAT}), decreases the viability of GBM cells with engineered expression of wild type (G432-IDH^{WT}) or mutant IDH (G432-IDH^{mut}), as well as GBM cells with spontaneous IDH mutations (G607, G809r).

FIG. 41 shows that treating mice using K90-114^{TAT}, but not K59-78^{TAT}, suppresses GBM growth (G411) and extends the survival of tumor-bearing mice.

FIG. 42 shows that treating mice using K90-114^{TAT}, but not K59-78^{TAT}, decreases proliferation, increases apoptosis, and reduces EAG2 expression of GBM cells in vivo.

FIG. 43 shows that treating mice using K90-114^{TAT} increases apoptosis of GBM cells in vivo. Note that K90-114^{TAT} selectively kills invasive GBM cells (arrows in FIG. 43) but not non-tumoral brain cells.

FIG. 44 shows that the growth of TMZ-resistant GBM cell lines (G411-TMZr, G532-TMZr, G799-TMZr, G800-TMZr) is suppressed by K90-114^{TAT}, but not K59-78^{TAT} or TMZ, treatment.

FIG. 45 shows that treating mice using K90-114^{TAT}, but not K59-78^{TAT}, suppresses the growth of TMZ-resistant GBM (G532-TMZr) in mice.

FIG. 46 shows that treatment using K90-114^{TAT}, but not K59-78^{TAT} or TMZ, extends the survival of mice bearing TMZ-resistant GBM (G532r-TMZr).

FIG. 47 depicts viability of G532 cells following exposure to various peptide treatments.

FIG. 48 depicts a schematic showing selection and establishment of TMZ-resistant cell lines.

FIG. 49 shows that treating mice with K90-114^{TAT}, but not K59-78^{TAT} or TMZ, decreased tumor cell proliferation, increased tumor cell apoptosis.

FIG. 50 presents quantified data corresponding to FIG. 49.

FIG. 51 shows reduced tumor burden and extension of mouse survival for animals treated with K90-114^{TAT}

FIG. 52 panel a and panel b show that K59-78TAT and K90-114TAT-treated mice displayed comparable body weight and survival.

FIG. 53 shows that internal organs (heart, kidney, liver, lung) did not reveal pathological features in mice treated with either peptide.

### DETAILED DESCRIPTION

Generally, the present disclosure provides a recombinant polypeptide comprising a polypeptide for preventing or reducing interaction between the human proteins EAG2 and Kvß2, and a cell-penetrating peptide. The polypeptide may comprise a portion of Kvß2 from or encompassing a region that is important for the interaction between the two proteins. The EAG2-Kvß2 complex is herein identified as a therapeutic target in certain cancers. Herein are provided therapeutic applications of the recombinant polypeptide in treatment of such cancers, including gliomas, such as low-grade gliomas that harbor IDH mutations or GBM, which may be recurrent and/or resistant to conventional treatment.

### Recombinant Polypeptides

In one aspect, there is provided a recombinant polypeptide comprising a) a polypeptide for preventing or reducing interaction between EAG2 and Kvß2, and b) a cell-penetrating peptide.

By “a polypeptide for preventing or reducing interaction between EAG2 and Kvß2” is meant a sequence of amino acids that prevents or reduces interaction between human voltage-gated potassium channel subfamily H member 5 (KCNH5, also known as EAG2) and human voltage-gated potassium channel subunit beta-2 (KCNAB2, also known as Kvß2). Example assays for assessing the interaction are described herein. EAG2 mRNA is represented by, for example, GenBank Accession No. NM_139318.5 (SEQ ID NO: 23). The EAG2 polypeptide is represented, for example, by GenBank Accession No. NP_647479.2 (SEQ ID NO: 24). The Kvß2 mRNA is represented by, for example, GenBank Accession No. NM_001199860.2 (SEQ ID NO: 25). The Kvß2 polypeptide is represented, for example, by GenBank Accession No. NP_001186789 (SEQ ID NO: 26).

By “preventing” will be understood a reduction in interaction that effectively reducing interaction to essentially undetectable levels, e.g., using the assays described herein.

By “reducing” will be understood a reduction in interaction as compared to an original level, e.g., measured in a cell prior to exposure to the recombinant polypeptide as described herein. It is to be understood that where “preventing or reducing” the interaction is referred to, corresponding embodiments are encompasses in each case in which the polypeptide is for reducing the interaction.

In one embodiment, the polypeptide for preventing or reducing interaction between EAG2 and Kvß2 comprises a fragment of Kvß2.

In one aspect, there is provided a recombinant polypeptide comprising a) a polypeptide for preventing or reducing interaction between EAG2 and Kvß2 comprising i) at least 5 contiguous amino acids from a region of human Kvß2 selected from the group consisting of amino acids 1 to 67 thereof, amino acids 90 to 114 thereof, and amino acids 343 to 355 thereof, or ii) a polypeptide that is at least 70% identical to i); and b) a cell-penetrating peptide.

By “contiguous” amino acids will be understood an uninterrupted portion of the amino acid sequence of human Kvß2.

In one embodiment, the polypeptide for preventing or reducing interaction between EAG2 and Kvß2 comprises at least 5 contiguous amino acid in the region of human Kvß2 from amino acids 1 to 67.

In one embodiment, the polypeptide for preventing or reducing interaction between EAG2 and Kvß2 comprises at least 5 contiguous amino acid in the region of human Kvß2 from amino acids 90 to 114.

In one embodiment, the polypeptide for preventing or reducing interaction between EAG2 and Kvß2 comprises at least 5 contiguous amino acid in the region of human Kvß2 from amino acids 343 to 355.

In one embodiment, the polypeptide for preventing or reducing interaction between EAG2 and Kvß2 comprises i) at least 6 contiguous amino acids from the region of Kvß2. In one embodiment, the polypeptide for preventing or reducing interaction between EAG2 and Kvß2 comprises i) at least 7 contiguous amino acids from the region of Kvß2. In one embodiment, the polypeptide for preventing or reducing interaction between EAG2 and Kvß2 comprises i) at least 8 contiguous amino acids from the region of Kvß2. In one embodiment, the polypeptide for preventing or reducing interaction between EAG2 and Kvß2 comprises i) at least 9 contiguous amino acids from the region of Kvß2. In one embodiment, the polypeptide for preventing or reducing interaction between EAG2 and Kvß2 comprises i) at least 10 contiguous amino acids from the region of Kvß2. In one embodiment, the polypeptide for preventing or reducing interaction between EAG2 and Kvß2 comprises i) at least 11 contiguous amino acids from the region of Kvß2. In one embodiment, the polypeptide for preventing or reducing interaction between EAG2 and Kvß2 comprises i) at least 12 contiguous amino acids from the region of Kvß2. In one embodiment, the polypeptide for preventing or reducing interaction between EAG2 and Kvß2 comprises i) at least 13 contiguous amino acids from the region of Kvß2. In one embodiment, the polypeptide for preventing or reducing interaction between EAG2 and Kvß2 comprises i) at least 14 contiguous amino acids from the region of Kvß2. In one embodiment, the polypeptide for preventing or reducing interaction between EAG2 and Kvß2 comprises i) at least 15 contiguous amino acids from the region of Kvß2. In one embodiment, the polypeptide for preventing or reducing interaction between EAG2 and Kvß2 comprises i) at least 16 contiguous amino acids from the region of Kvß2. In one embodiment, the polypeptide for preventing or reducing interaction between EAG2 and Kvß2 comprises i) at least 17 contiguous amino acids from the region of Kvß2. In one embodiment, the polypeptide for preventing or reducing interaction between EAG2 and Kvß2 comprises i) at least 18 contiguous amino acids from the region of Kvß2. In one embodiment, the polypeptide for preventing or reducing interaction between EAG2 and Kvß2 comprises i) at least 19 contiguous amino acids from the region of Kvß2. In one embodiment, the polypeptide for preventing or reducing interaction between EAG2 and Kvß2 comprises i) at least 20 contiguous amino acids from the region of Kvß2. In one embodiment, the polypeptide for preventing or reducing interaction between EAG2 and Kvß2 comprises i) at least 21 contiguous amino acids from the region of Kvß2. In one embodiment, the polypeptide for preventing or reducing interaction between EAG2 and Kvß2 comprises i) at least 22 contiguous amino acids from the region of Kvß2. In one embodiment, the polypeptide for preventing or reducing interaction between EAG2 and Kvß2 comprises i) at least 23 contiguous amino acids from the region of Kvß2. In one embodiment, the polypeptide for preventing or reducing interaction between EAG2 and Kvß2 comprises i) at least 24 contiguous amino acids from the region of Kvß2. In one embodiment, the polypeptide for preventing or reducing interaction between EAG2 and Kvß2 comprises i) at least 25 contiguous amino acids from the region of Kvß2. In one embodiment, the polypeptide for preventing or reducing interaction between EAG2 and Kvß2 comprises i) at least 26 contiguous amino acids from the region of Kvß2. In one embodiment, the polypeptide for preventing or reducing interaction between EAG2 and Kvß2 comprises i) at least 27 contiguous amino acids from the region of Kvß2. In one embodiment, the polypeptide for preventing or reducing interaction between EAG2 and Kvß2 comprises i) at least 28 contiguous amino acids from the region of Kvß2. In one embodiment, the polypeptide for preventing or reducing interaction between EAG2 and Kvß2 comprises i) at least 29 contiguous amino acids from the region of Kvß2. In one embodiment, the polypeptide for preventing or reducing interaction between EAG2 and Kvß2 comprises i) at least 30 contiguous amino acids from the region of Kvß2. In one embodiment, the polypeptide for preventing or reducing interaction between EAG2 and Kvß2 comprises i) at least 31 contiguous amino acids from the region of Kvß2. In one embodiment, the polypeptide for preventing or reducing interaction between EAG2 and Kvß2 comprises i) at least 32 contiguous amino acids from the region of Kvß2. In one embodiment, the polypeptide for preventing or reducing interaction between EAG2 and Kvß2 comprises i) at least 33 contiguous amino acids from the region of Kvß2. In one embodiment, the polypeptide for preventing or reducing interaction between EAG2 and Kvß2 comprises i) at least 34 contiguous amino acids from the region of Kvß2. In one embodiment, the polypeptide for preventing or reducing interaction between EAG2 and Kvß2 comprises i) at least 35 contiguous amino acids from the region of Kvß2. In one embodiment, the polypeptide for preventing or reducing interaction between EAG2 and Kvß2 comprises i) at least 36 contiguous amino acids from the region of Kvß2. In one embodiment, the polypeptide for preventing or reducing interaction between EAG2 and Kvß2 comprises i) at least 37 contiguous amino acids from the region of Kvß2. In one embodiment, the polypeptide for preventing or reducing interaction between EAG2 and Kvß2 comprises i) at least 38 contiguous amino acids from the region of Kvß2. In one embodiment, the polypeptide for preventing or reducing interaction between EAG2 and Kvß2 comprises i) at least 39 contiguous amino acids from the region of Kvß2. In one embodiment, the polypeptide for preventing or reducing interaction between EAG2 and Kvß2 comprises i) at least 40 contiguous amino acids from the region of Kvß2.

In one embodiment, the polypeptide for preventing or reducing interaction between EAG2 and Kvß2 is a polypeptide that reduces interaction between EAG2 and Kvß2.

In one embodiment, the polypeptide for preventing or reducing interaction between EAG2 and Kvß2 is as defined in ii).

In one embodiment, the polypeptide for preventing or reducing interaction between EAG2 and Kvß2 is at least 80% identical to the polypeptide of i).

In one embodiment, the polypeptide for preventing or reducing interaction between EAG2 and Kvß2 is at least 85% identical to the polypeptide of i).

In one embodiment, the polypeptide for preventing or reducing interaction between EAG2 and Kvß2 is at least 90% identical to the polypeptide of i).

In one embodiment, the polypeptide for preventing or reducing interaction between EAG2 and Kvß2 is at least 95% identical to the polypeptide of i).

In one embodiment, the polypeptide for preventing or reducing interaction between EAG2 and Kvß2 is as defined in i).

In one aspect, there is provided a recombinant polypeptide comprising: a) a polypeptide for preventing or reducing interaction between EAG2 and Kvß2 comprising: i) a contiguous portion of human Kvß2 encompassing at least amino acids 90 to 114 thereof, or ii) a polypeptide that is at least 70% identical to i); and b) a cell-penetrating peptide.

In one embodiment, the polypeptide for preventing or reducing interaction between EAG2 and Kvß2 comprises i) at least 1 additional contiguous amino acid from Kvß2. In one embodiment, the polypeptide for preventing or reducing interaction between EAG2 and Kvß2 comprises i) at least 2 additional contiguous amino acids from Kvß2. In one embodiment, the polypeptide for preventing or reducing interaction between EAG2 and Kvß2 comprises i) at least 3 additional contiguous amino acids from Kvß2. In one embodiment, the polypeptide for preventing or reducing interaction between EAG2 and Kvß2 comprises i) at least 4 additional contiguous amino acids from Kvß2. In one embodiment, the polypeptide for preventing or reducing interaction between EAG2 and Kvß2 comprises i) at least 5 additional contiguous amino acids from Kvß2. In one embodiment, the polypeptide for preventing or reducing interaction between EAG2 and Kvß2 comprises i) at least 6 additional contiguous amino acids from Kvß2. In one embodiment, the polypeptide for preventing or reducing interaction between EAG2 and Kvß2 comprises i) at least 7 additional contiguous amino acids from Kvß2. In one embodiment, the polypeptide for preventing or reducing interaction between EAG2 and Kvß2 comprises i) at least 8 additional contiguous amino acids from Kvß2. In one embodiment, the polypeptide for preventing or reducing interaction between EAG2 and Kvß2 comprises i) at least 9 additional contiguous amino acids from Kvß2. In one embodiment, the polypeptide for preventing or reducing interaction between EAG2 and Kvß2 comprises i) at least 10 additional contiguous amino acids from Kvß2. In one embodiment, the polypeptide for preventing or reducing interaction between EAG2 and Kvß2 comprises i) at least 11 additional contiguous amino acids from Kvß2. In one embodiment, the polypeptide for preventing or reducing interaction between EAG2 and Kvß2 comprises i) at least 12 additional contiguous amino acids from Kvß2. In one embodiment, the polypeptide for preventing or reducing interaction between EAG2 and Kvß2 comprises i) at least 13 additional contiguous amino acids from Kvß2. In one embodiment, the polypeptide for preventing or reducing interaction between EAG2 and Kvß2 comprises i) at least 14 additional contiguous amino acids from Kvß2. In one embodiment, the polypeptide for preventing or reducing interaction between EAG2 and Kvß2 comprises i) at least 15 additional contiguous amino acids from Kvß2. In one embodiment, the polypeptide for preventing or reducing interaction between EAG2 and Kvß2 comprises i) at least 16 additional contiguous amino acids from Kvß2. In one embodiment, the polypeptide for preventing or reducing interaction between EAG2 and Kvß2 comprises i) at least 17 additional contiguous amino acids from Kvß2. In one embodiment, the polypeptide for preventing or reducing interaction between EAG2 and Kvß2 comprises i) at least 18 additional contiguous amino acids from Kvß2. In one embodiment, the polypeptide for preventing or reducing interaction between EAG2 and Kvß2 comprises i) at least 19 additional contiguous amino acids from Kvß2. In one embodiment, the polypeptide for preventing or reducing interaction between EAG2 and Kvß2 comprises i) at least 20 additional contiguous amino acids from Kvß2.

In one embodiment, the polypeptide for preventing or reducing interaction between EAG2 and Kvß2 is a polypeptide that reduces interaction between EAG2 and Kvß2.

In one embodiment, the polypeptide for preventing or reducing interaction between EAG2 and Kvß2 is as defined in ii).

In one embodiment, the polypeptide as defined in ii) is at least 80% identical to the polypeptide as defined in i).

In one embodiment, the polypeptide as defined in ii) is at least 85% identical to the polypeptide as defined in i).

In one embodiment, the polypeptide as defined in ii) is at least 90% identical to the polypeptide as defined in i).

In one embodiment, the polypeptide as defined in ii) is at least 95% identical to the polypeptide as defined in i).

In one embodiment, the polypeptide for preventing or reducing interaction between EAG2 and Kvß2 is as defined in i).

In one embodiment, the polypeptide for preventing or reducing the interaction between EAG2 and Kvß2 comprises amino acids 90 to 114 of human Kvß2 (SEQ ID NO: 1).

In one embodiment, the polypeptide for preventing or reducing the interaction between EAG2 and Kvß2 consists of amino acids 90 to 114 of human Kvß2 (SEQ ID NO: 1).

Example polypeptides for preventing or reducing the interaction between EAG2 and Kvß2, according to particular embodiments, are provided in Table 1. There are herein terms “designer interference polypeptides” or “DIPs”.

TABLE 1
Example Designer Interference
Peptides (DIPs)

| SEQ ID | | |
|---|---|---|
| No. | Name | Sequence |
| 1 | Designer | YAAGKAE |
| | interference | VVLGNII |
| | peptide (DIP) | KKKGWRR |
| | | SSLV |
| 2 | DIP C-terminal | YAAGKAE |
| | truncation | VVLGNII |
| | | KKK |
| 3 | DIP N-terminal | KAEVVLG |
| | truncation | NIIKKKG |
| | | WRRSS |
| 4 | DIP Dual N-/C- | KAEVVLG |
| | truncation | NIIKKK |

*D-amino acids

In one embodiment, the polypeptide for preventing or reducing interaction between EAG2 and Kvß2 comprises a C-terminal truncation of SEQ ID NO: 1. In one embodiment, the polypeptide for preventing or reducing interaction between EAG2 and Kvß2 comprises amino acids having SEQ ID NO: 2. In one embodiment, the polypeptide for preventing or reducing interaction between EAG2 and Kvß2 comprises amino acids that are at least 80% identical to SEQ ID NO: 2. In one embodiment, the polypeptide for preventing or reducing interaction between EAG2 and Kvß2 comprises amino acids that are at least 90% identical to SEQ ID NO: 2.

In one embodiment, the polypeptide for preventing or reducing interaction between EAG2 and Kvß2 comprises an N-terminal truncation of SEQ ID NO: 1. In one embodiment, the polypeptide for preventing or reducing interaction between EAG2 and Kvß2 comprises amino acids having SEQ ID NO: 3. In one embodiment, the polypeptide for preventing or reducing interaction between EAG2 and Kvß2 comprises amino acids that are at least 80% identical to SEQ ID NO: 3. In one embodiment, the polypeptide for preventing or reducing interaction between EAG2 and Kvß2 comprises amino acids that are at least 90% identical to SEQ ID NO: 3.

In one embodiment, the polypeptide for preventing or reducing interaction between EAG2 and Kvß2 comprises both N- and C-terminal truncations of SEQ ID NO: 1. In one embodiment, the polypeptide for preventing or reducing interaction between EAG2 and Kvß2 comprises amino acids having SEQ ID NO: 4. In one embodiment, the polypeptide for preventing or reducing interaction between EAG2 and Kvß2 comprises amino acids that are at least 80% identical to SEQ ID NO: 4. In one embodiment, the polypeptide for preventing or reducing interaction between EAG2 and Kvß2 comprises amino acids that are at least 90% identical to SEQ ID NO: 5.

By “cell-penetrating peptide” (CPP) is meant a sequence of amino acids that allows, facilitates, or otherwise promotes its own entry, and the entry of a linked polypeptides, into a cell. Example of cell-penetrating peptides are set forth in Table 2.

TABLE 2
Example Cell-penetrating Peptides (CPPs)

| | SEQ ID | |
|---|---|---|
| CPP | NO. | Sequence |
| HIV TAT | 5 | GRKKRRQRRRPQ |
| Circularizable | 6 | CGRKKRRQRRRPQ |
| HIV TAT | | C |
| HA-TAT | 7 | GDIMGEWGNEIFG |
| | | AIAGFLGGRKKRR |
| | | QRRRPQ |
| Penetratin | 8 | RQIKIWFQNRRMK |
| | | WKK |
| Circularizable | 9 | CRQIKIWFQNRRM |
| Penetratin | | KWKKC |
| Transportan | 10 | GWTLNSAGYLLGK |
| | | INLKALAALAKKI |
| | | L |
| Circularizable | 11 | CGWTLNSAGYLLG |
| Transportan | | KINLKALAALAKK |
| | | ILC |
| Xentry | 12 | LCLRPVG |
| Circularizable | 13 | CLCLRPVGC |
| Xentry | | |
| R8 | 14 | RRRRRRRR |
| Circularizable R8 | 15 | CRRRRRRRRC |

The cell-penetrating peptide may be positioned N- or C-terminally with respect to the polypeptide for preventing or reducing interaction between EAG2 and Kvß2. In one embodiment, the cell-penetrating peptide is positioned at the N-terminus of the recombinant polypeptide. In one embodiment, the cell-penetrating peptide is positioned at the C-terminus of the recombinant polypeptide.

In one embodiment, the cell-penetrating peptide is selected from the group consisting of:
HIV TAT (SEQ ID NO: 5),
Circularizable HIV TAT (SEQ ID NO: 6),
HA-TAT (SEQ ID NO: 7),
Penetratin (SEQ ID NO: 8),
Circularizable Penetratin (SEQ ID NO: 9),
Transportan (SEQ ID NO: 10),
Circularizable Transportan (SEQ ID NO: 11),
Xentry (SEQ ID NO: 12),
Circularizable Xentry (SEQ ID NO: 13),
R8 (SEQ ID NO: 14),
Circularizable R8 (SEQ ID NO: 15); and
a sequence that is at least 80% identical thereto to one of the foregoing across the full length thereof.

In one embodiment, the cell-penetrating peptide is selected from the group consisting of:
HIV TAT (SEQ ID NO: 5),
Circularizable HIV TAT (SEQ ID NO: 6),
HA-TAT (SEQ ID NO: 7),
Penetratin (SEQ ID NO: 8),
Circularizable Penetratin (SEQ ID NO: 9),
Transportan (SEQ ID NO: 10),
Circularizable Transportan (SEQ ID NO: 11),
Xentry (SEQ ID NO: 12),
Circularizable Xentry (SEQ ID NO: 13),
R8 (SEQ ID NO: 14),
Circularizable R8 (SEQ ID NO: 15); and
a sequence that is at least 90% identical thereto to one of the foregoing across the full length thereof.

In one embodiment, the cell-penetrating peptide is selected from the group consisting of:
HIV TAT (SEQ ID NO: 5),
Circularizable HIV TAT (SEQ ID NO: 6),
HA-TAT (SEQ ID NO: 7),
Penetratin (SEQ ID NO: 8),
Circularizable Penetratin (SEQ ID NO: 9),
Transportan (SEQ ID NO: 10),
Circularizable Transportan (SEQ ID NO: 11),
Xentry (SEQ ID NO: 12),
Circularizable Xentry (SEQ ID NO: 13),
R8 (SEQ ID NO: 14), and
Circularizable R8 (SEQ ID NO: 15).

In one embodiment, the cell-penetrating peptide is selected from the group consisting of:
HIV TAT (SEQ ID NO: 5),
Circularizable HIV TAT (SEQ ID NO: 6),
HA-TAT (SEQ ID NO: 7),
Penetratin (SEQ ID NO: 8),
Circularizable Penetratin (SEQ ID NO: 9),
Transportan (SEQ ID NO: 10),
Circularizable Transportan (SEQ ID NO: 11). and
a sequence that is at least 80% identical thereto to one of the foregoing across the full length thereof.

In one embodiment, the cell-penetrating peptide is selected from the group consisting of:
HIV TAT (SEQ ID NO: 5),
Circularizable HIV TAT (SEQ ID NO: 6),
HA-TAT (SEQ ID NO: 7),
Penetratin (SEQ ID NO: 8),
Circularizable Penetratin (SEQ ID NO: 9),
Transportan (SEQ ID NO: 10),
Circularizable Transportan (SEQ ID NO: 11). and
a sequence that is at least 90% identical thereto to one of the foregoing across the full length thereof.

In one embodiment, the cell-penetrating peptide is selected from the group consisting of:
HIV TAT (SEQ ID NO: 5),
Circularizable HIV TAT (SEQ ID NO: 6),
HA-TAT (SEQ ID NO: 7),
Penetratin (SEQ ID NO: 8),
Circularizable Penetratin (SEQ ID NO: 9),
Transportan (SEQ ID NO: 10), and
Circularizable Transportan (SEQ ID NO: 11).

In one embodiment, the cell-penetrating peptide is selected from the group consisting of:
HIV TAT (SEQ ID NO: 5),
Penetratin (SEQ ID NO: 8), and
a sequence that is at least 80% identical thereto to one of the foregoing across the full length thereof.

In one embodiment, the cell-penetrating peptide is selected from the group consisting of:
HIV TAT (SEQ ID NO: 5),
Penetratin (SEQ ID NO: 8), and
a sequence that is at least 90% identical thereto to one of the foregoing across the full length thereof.

In one embodiment, the cell-penetrating peptide is selected from the group consisting of:
HIV TAT (SEQ ID NO: 5), and
Penetratin (SEQ ID NO: 8).

In one embodiment, the cell-penetrating peptide is HIV TAT (SEQ ID NO: 5).

In one embodiment, the cell-penetrating peptide is Circularizable HIV TAT (SEQ ID NO: 6).

In one embodiment, the cell-penetrating peptide is HA-TAT (SEQ ID NO: 7).

In one embodiment, the cell-penetrating peptide is Penetratin (SEQ ID NO: 8).

In one embodiment, the cell-penetrating peptide is Circularizable Penetratin (SEQ ID NO: 9).

In one embodiment, the cell-penetrating peptide is Transportan (SEQ ID NO: 10).

In one embodiment, the cell-penetrating peptide is Circularizable Transportan (SEQ ID NO: 11).

In one embodiment, the cell-penetrating peptide is Xentry (SEQ ID NO: 12).

In one embodiment, the cell-penetrating peptide is Circularizable Xentry (SEQ ID NO: 13).

In one embodiment, the cell-penetrating peptide is R8 (SEQ ID NO: 14).

In one embodiment, the cell-penetrating peptide is Circularizable R8 (SEQ ID NO: 15).

In one embodiment, the cell-penetrating peptide is positioned N-terminally with respect to the polypeptide for preventing or reducing interaction between EAG2 and Kvß2. In one embodiment, the cell-penetrating peptide is positioned at the N-terminus of the recombinant polypeptide.

In one embodiment, the cell-penetrating peptide is positioned C-terminally with respect to the polypeptide for preventing or reducing interaction between EAG2 and Kvß2. In one embodiment, the cell-penetrating peptide is positioned at the C-terminus of the recombinant polypeptide.

In one embodiment, the CPP comprises HIV TAT (SEQ ID NO: 5) or a sequence that is at least 80% identical thereto over the full length thereof. In one embodiment, the CPP comprises HIV TAT (SEQ ID NO: 5) or a sequence that is at least 90% identical thereto over the full length thereof. In one embodiment, the CPP comprises SEQ ID NO: 5. In one embodiment, the CPP consists of SEQ ID NO: 5. In one embodiment, the HIV TAT is positioned N-terminally with respect to the polypeptide for preventing or reducing interaction between EAG2 and Kvß2. In one embodiment, the HIV TAT is positioned at the N-terminus of the recombinant polypeptide.

In one embodiment, the CPP comprises circularizable HIV TAT (SEQ ID NO: 6) or a sequence that is at least 80% identical thereto over the full length thereof. In one embodiment, the CPP comprises circularizable HIV TAT (SEQ ID NO: 6) or a sequence that is at least 90% identical thereto over the full length thereof. In one embodiment, the CPP comprises amino acids of SEQ ID NO: 6. In one embodiment, the CPP consists of amino acids of SEQ ID NO: 6. In one embodiment, the circularizable HIV TAT is positioned N-terminally with respect to the polypeptide for preventing or reducing interaction between EAG2 and Kvß2. In one embodiment, the circularizable HIV TAT is positioned at the N-terminus of the recombinant polypeptide.

In one embodiment, the CPP comprises HA-TAT (SEQ ID NO: 7) or a sequence that is at least 80% identical thereto over the full length thereof. In one embodiment, the CPP comprises HA-TAT (SEQ ID NO: 7) or a sequence that is at least 90% identical thereto over the full length thereof. In one embodiment, the CPP comprises amino acids of SEQ ID NO: 7. In one embodiment, the CPP consists of amino acids of SEQ ID NO: 7. In one embodiment, the HA-TAT is positioned N-terminally with respect to the polypeptide for preventing or reducing interaction between EAG2 and Kvß2. In one embodiment, the HA-TAT is positioned at the N-terminus of the recombinant polypeptide.

In one embodiment, the CPP comprises Penetratin (SEQ ID NO: 8) or a sequence that is at least 80% identical thereto over the full length thereof. In one embodiment, the CPP comprises Penetratin (SEQ ID NO: 8) or a sequence that is at least 90% identical thereto over the full length thereof. In one embodiment, the CPP comprises amino acids of SEQ ID NO: 8. In one embodiment, the CPP consists of amino acids of SEQ ID NO: 8. In one embodiment, the Penetratin is positioned N-terminally with respect to the polypeptide for preventing or reducing interaction between EAG2 and Kvß2. In one embodiment, the Penetratin is positioned at the N-terminus of the recombinant polypeptide.

In one embodiment, the CPP comprises circularizable Penetratin (SEQ ID NO: 9) or a sequence that is at least 80% identical thereto over the full length thereof. In one embodiment, the CPP comprises circularizable Penetratin (SEQ ID NO: 9) or a sequence that is at least 90% identical thereto over the full length thereof. In one embodiment, the CPP comprises amino acids of SEQ ID NO: 9. In one embodiment, the CPP consists of amino acids of SEQ ID NO: 9. In one embodiment, the circularizable Penetratin is positioned N-terminally with respect to the polypeptide for preventing or reducing interaction between EAG2 and Kvß2. In one embodiment, the circularizable Penetratin is positioned at the N-terminus of the recombinant polypeptide.

In one embodiment, the CPP comprises Transportan (SEQ ID NO: 10) or a sequence that is at least 80% identical thereto over the full length thereof. In one embodiment, the CPP comprises Transportan (SEQ ID NO: 10) or a sequence that is at least 90% identical thereto over the full length thereof. In one embodiment, the CPP comprises amino acids of SEQ ID NO: 10. In one embodiment, the CPP consists of amino acids of SEQ ID NO: 10. In one embodiment, the Transportan is positioned N-terminally with respect to the polypeptide for preventing or reducing interaction between EAG2 and Kvß2. In one embodiment, the Penetratin is positioned at the N-terminus of the recombinant polypeptide.

In one embodiment, the CPP comprises circularizable Transportan (SEQ ID NO: 11) or a sequence that is at least 80% identical thereto over the full length thereof. In one embodiment, the CPP comprises circularizable Transportan (SEQ ID NO: 11) or a sequence that is at least 90% identical thereto over the full length thereof. In one embodiment, the CPP comprises amino acids of SEQ ID NO: 11. In one embodiment, the CPP consists of amino acids of SEQ ID NO: 11. In one embodiment, the circularizable Transportan is positioned N-terminally with respect to the polypeptide for preventing or reducing interaction between EAG2 and Kvß2. In one embodiment, the circularizable Transportan is positioned at the N-terminus of the recombinant polypeptide.

In one embodiment, the CPP comprises Xentry (SEQ ID NO: 12) or a sequence that is at least 80% identical thereto over the full length thereof. In one embodiment, the CPP comprises Xentry (SEQ ID NO: 12) or a sequence that is at least 90% identical thereto over the full length thereof. In one embodiment, the CPP comprises amino acids of SEQ ID NO: 12. In one embodiment, the CPP consists of amino acids of SEQ ID NO: 12. In one embodiment, the Xentry is positioned N-terminally with respect to the polypeptide for preventing or reducing interaction between EAG2 and Kvß2. In one embodiment, the Xentry is positioned at the N-terminus of the recombinant polypeptide.

In one embodiment, the CPP comprises circularizable Xentry (SEQ ID NO: 13) or a sequence that is at least 80% identical thereto over the full length thereof. In one embodiment, the CPP comprises circularizable Xentry (SEQ ID NO: 13) or a sequence that is at least 90% identical thereto over the full length thereof. In one embodiment, the CPP comprises amino acids of SEQ ID NO: 13. In one embodiment, the CPP consists of amino acids of SEQ ID NO: 13. In one embodiment, the circularizable Xentry is positioned N-terminally with respect to the polypeptide for preventing or reducing interaction between EAG2 and Kvß2. In one embodiment, the circularizable Xentry is positioned at the N-terminus of the recombinant polypeptide.

In one embodiment, the CPP comprises R8 (SEQ ID NO: 14) or a sequence that is at least 80% identical thereto over the full length thereof. In one embodiment, the CPP comprises R8 (SEQ ID NO: 14) or a sequence that is at least 90% identical thereto over the full length thereof. In one embodiment, the CPP comprises amino acids of SEQ ID NO: 14. In one embodiment, the CPP consists of amino acids of SEQ ID NO: 14. In one embodiment, the R8 is positioned N-terminally with respect to the polypeptide for preventing or reducing interaction between EAG2 and Kvß2. In one embodiment, the R8 is positioned at the N-terminus of the recombinant polypeptide.

In one embodiment, the CPP comprises circularizable R8 (SEQ ID NO: 15) or a sequence that is at least 80% identical thereto over the full length thereof. In one embodiment, the CPP comprises circularizable R8 (SEQ ID NO: 15) or a sequence that is at least 90% identical thereto over the full length thereof. In one embodiment, the CPP comprises amino acids of SEQ ID NO: 15. In one embodiment, the CPP consists of amino acids of SEQ ID NO: 15. In one embodiment, the circularizable R8 is positioned N-terminally with respect to the polypeptide for preventing or reducing interaction between EAG2 and Kvß12. In one embodiment, the circularizable R8 is positioned at the N-terminus of the recombinant polypeptide.

Example recombinant polypeptides, according to particular embodiments are provided in Table 3.

TABLE 3
Example Recombinant Polypeptides and Linker

| | | Sequence |
|---|---|---|
| SEQ | | (linker |
| ID | | underlined; |
| No. | Name | DIP bolded) |
| 16 | Linker | GSGSGS |
| 17 | TAT-DIP (also | GRKKRRQRRR |
| | termed | PQGSGSGSYA |
| | K190-114TAT) | AGKAEVVLGN |
| | | IIKKKGWRRS |
| | | SLV |
| 18 | CTAT-DIP (with | [C]GRKKRRQ |
| | circularized | RRRPQ[C]GS |
| | TAT linked at | GSGSYAAG |
| | cysteine | KAEVVLGNII |
| | residues | KKKGWRRSSL |
| | denoted | V |
| | by [C]) | |
| 19 | TAT-DIP mod#1 | GRKKRRQRRR |
| | (C-term | PQGSGSGSYA |
| | truncation) | AGKAEVVLGN |
| | | IIKKK |
| 20 | TAT-DIP mod#2 | GRKKRRQRRR |
| | (N-term | PQGSGSGSKA |
| | truncation) | EVVLGNIIKK |
| | | KGWRRSS |
| 21 | R8-DIP | RRRRRRRRKA |
| | | EVVLGNIIKK |
| | | K |
| 22 | retro-inverso | VLSSRRWGKK |
| | TAT-DIP | KIINGLVVEA |
| | | KGAAYSGSGS |
| | | GQPRRRQRRK |
| | | KRG* |

In one embodiment of each of the above, the linker (underlined above) is absent.

In one embodiment, the recombinant polypeptide for preventing or reducing the interaction between EAG2 and Kvß2 is spaced apart from the cell-penetrating peptide by an amino acid linker. The amino acid linker may comprise, for example at least 1, 2, 3, 4, 5, 6, 7, 8, 9, or 10 amino acid residues. In one example embodiment, the amino acid linker comprises amino acids GSGSGS (SEQ ID NO: 16).

In one embodiment, the recombinant polypeptide comprises amino acids that are at least 70% identical to SEQ ID NO: 17. In one embodiment, the recombinant polypeptide comprises amino acids that are at least 80% identical to SEQ ID NO: 17. In one embodiment, the recombinant polypeptide comprises amino acids that are at least 90% identical to SEQ ID NO: 17. In one embodiment, the recombinant polypeptide comprises amino acids that are at least 95% identical to SEQ ID NO: 17. In one embodiment, the recombinant polypeptide comprises amino acids having SEQ ID NO: 17. In one embodiment, the recombinant polypeptide consists of amino acids having SEQ ID NO: 17.

In one embodiment, the recombinant polypeptide comprises amino acids that are at least 70% identical to SEQ ID NO: excluding the linker. In one embodiment, the recombinant polypeptide comprises amino acids that are at least 80% identical to SEQ ID NO: 17 excluding the linker. In one embodiment, the recombinant polypeptide comprises amino acids that are at least 90% identical to SEQ ID NO: 17 excluding the linker. In one embodiment, the recombinant polypeptide comprises amino acids that are at least 95% identical to SEQ ID NO: 17 excluding the linker. In one embodiment, the recombinant polypeptide comprises amino acids having SEQ ID NO: 17 excluding the linker. In one embodiment, the recombinant polypeptide consists of amino acids having SEQ ID NO: 17 excluding the linker.

In one embodiment, the recombinant polypeptide comprises amino acids that are at least 70% identical to SEQ ID NO: 18. In one embodiment, the recombinant polypeptide comprises amino acids that are at least 80% identical to SEQ ID NO: 18. In one embodiment, the recombinant polypeptide comprises amino acids that are at least 90% identical to SEQ ID NO: 18. In one embodiment, the recombinant polypeptide comprises amino acids that are at least 95% identical to SEQ ID NO: 18. In one embodiment, the recombinant polypeptide comprises amino acids having SEQ ID NO: 18. In one embodiment, the recombinant polypeptide consists of amino acids having SEQ ID NO: 18.

In one embodiment, the recombinant polypeptide comprises amino acids that are at least 70% identical to SEQ ID NO: 19. In one embodiment, the recombinant polypeptide comprises amino acids that are at least 80% identical to SEQ ID NO: 19. In one embodiment, the recombinant polypeptide comprises amino acids that are at least 90% identical to SEQ ID NO: 19. In one embodiment, the recombinant polypeptide comprises amino acids that are at least 95% identical to SEQ ID NO: 19. In one embodiment, the recombinant polypeptide comprises amino acids having SEQ ID NO: 19. In one embodiment, the recombinant polypeptide consists of amino acids having SEQ ID NO: 19.

In one embodiment, the recombinant polypeptide comprises amino acids that are at least 70% identical to SEQ ID NO: 20. In one embodiment, the recombinant polypeptide comprises amino acids that are at least 80% identical to SEQ ID NO: 20. In one embodiment, the recombinant polypeptide comprises amino acids that are at least 90% identical to SEQ ID NO: 20. In one embodiment, the recombinant polypeptide comprises amino acids that are at least 95% identical to SEQ ID NO: 20. In one embodiment, the recombinant polypeptide comprises amino acids having SEQ ID NO: 20. In one embodiment, the recombinant polypeptide consists of amino acids having SEQ ID NO: 20.

In one embodiment, the recombinant polypeptide comprises amino acids that are at least 70% identical to SEQ ID NO: 21. In one embodiment, the recombinant polypeptide comprises amino acids that are at least 80% identical to SEQ ID NO: 21. In one embodiment, the recombinant polypeptide comprises amino acids that are at least 90% identical to SEQ ID NO: 21. In one embodiment, the recombinant polypeptide comprises amino acids that are at least 95% identical to SEQ ID NO: 21. In one embodiment, the recombinant polypeptide comprises amino acids having SEQ ID NO: 21. In one embodiment, the recombinant polypeptide consists of amino acids having SEQ ID NO: 21.

In one aspect, there is provided a retro-inverso polypeptide based on any one of the recombinant polypeptides described herein. By “retro-inverso” and “based on” will be understood a polypeptide comprising D-amino acids in reverse order to the sequence of a reference polypeptide comprising L-amino acids. In one embodiment, the retro-inverso polypeptide comprises D-amino acids in an order reverse to that of amino acids of any one of the recombinant polypeptide as defined herein. In one embodiment, the D-amino acids comprise the sequence of amino acid positions 1 to 25 of SEQ ID NO: 22. In one embodiment, the D-amino acids are at least 70% identical to SEQ ID NO: 22. In one embodiment, the D-amino acids are at least 80% identical to SEQ ID NO: 22. In one embodiment, the D-amino acids are at least 90% identical to SEQ ID NO: 22. In one embodiment, the D-amino acids are at least 95% identical to SEQ ID NO: 22. In one embodiment, the D-amino acids comprise the sequence of SEQ ID NO: 22. In one embodiment, the D-amino acids consist of the sequence of SEQ ID NO: 22.

Where percent identifies are discussed herein, it will be appreciated that these are stated in respect of an alignment across the full length of a particular reference sequence.

Where sequences differ from references sequences, in some embodiments these sequence differences will be conservative amino acid substitutions. Conservative amino acid substitutions are substitutions in which amino acids of a particular class are substituted with another amino acid of the same class. Classes include aliphatic amino acids (G, A, V, L, and I), aromatic amino acids (S, C, U, T, and M), cyclic amino acids (P), basic amino acids (H, K, and R), and acidic amino acids and their amides (D, E, N, and Q). Polypeptides bearing such substitutions could be tested for activity using assays described herein.

### Nucleic Acids and Vectors

In one aspect, there is provided a nucleic acid encoding the recombinant polypeptide as herein described.

In one aspect, there is provided a vector comprising the nucleic acid as herein described.

### Host Cells

In one aspect, there is a provided a host cell comprising the nucleic acid as herein described or the vector as herein described.

### Compositions

In one aspect, there is provided a composition comprising the recombinant polypeptide as herein described, the nucleic acid as herein described, or the vector as herein described; together with an excipient, diluent, or carrier.

In one aspect, there is provided a pharmaceutical composition comprising a recombinant polypeptide as herein described, the nucleic acid as herein described, or the vector as herein described; together with a pharmaceutically acceptable excipient, diluent, or carrier.

### Methods and Uses

In one aspect, there is provided a method of preventing or reducing interaction of EAG2 and Kvß2 in a cell comprising: contacting the cell with the recombinant polypeptide as herein described, the nucleic acid as herein described, the vector as herein described, or the pharmaceutical composition as described herein.

In one aspect, there is provided a use of the recombinant polypeptide as herein described, the nucleic acid as herein described, the vector as herein described, or the pharmaceutical composition as described herein for preventing or reducing interaction of EAG2 and Kvß2 in a cell.

In one aspect, there is provided a use of the recombinant polypeptide as herein described, the nucleic acid as herein described, the vector as herein described, or the pharmaceutical composition as described herein for preparation of a medicament for preventing or reducing interaction of EAG2 and Kvß2 in a cell.

In one aspect, there is provided the recombinant polypeptide as herein described, the nucleic acid as herein described, the vector as herein described, or the pharmaceutical composition as described herein for use in preventing or reducing interaction of EAG2 and Kvß2 in a cell.

In one aspect, there is provided a method of treating cancer in a subject comprising: administering to the subject the recombinant polypeptide as herein described, the nucleic acid as herein described, the vector as herein described, or the pharmaceutical composition as described herein.

In one aspect, there is provided a use of the recombinant polypeptide as herein described, the nucleic acid as herein described, the vector as herein described, or the pharmaceutical composition as described herein for treatment of cancer in a subject.

In one aspect, there is provided a use of the recombinant polypeptide as herein described, the nucleic acid as herein described, the vector as herein described, or the pharmaceutical composition as described herein for preparation of a medicament for treatment of cancer in a subject.

In one aspect, there is provided the recombinant polypeptide as herein described, the nucleic acid as herein described, the vector as herein described, or the pharmaceutical composition as described herein for use in treatment of cancer in a subject.

In one embodiment, the cancer is a glioma or a medulloblastoma. In one embodiment, the cancer is a glioma. In one embodiment, the glioma is a grade 1 glioma. In one embodiment, the glioma is a grade 2 glioma. In one embodiment, the glioma is a grade 3 glioma. In one embodiment, the glioma is a grade 4 glioma, which is also known as glioblastoma (GBM). In one embodiment the GBM is an IDH wild type GBM. In one embodiment, the GBM is an IDH mutant GBM.

In one embodiment, the cancer is radiation resistant, chemotherapy resistant, targeted therapy-resistant.

By “targeted therapy” is meant any cancer therapy that uses drugs or other chemicals which target specific genes or protein that are oncogenic or otherwise involved or associated with cancer pathology.

In one embodiment, the cancer is resistant to TMZ.

In one embodiment, the cancer is a post-therapy or recurrent.

### Screening Platform

In one aspect, there is provided a method of screening for a candidate therapeutic for cancer comprising: contacting a human cell with a test compound, wherein the human cell has an original level of interaction between EAG2 and Kvß2, measuring a level of interaction between EAG2 between Kvß2 after the step of contacting, and identifying the test compound as a candidate therapeutic for cancer if the measured level of the interaction between EAG2 and Kvß2 is reduced compared to the original level.

In one embodiment, the cancer is a glioma or a medulloblastoma. In one embodiment, the cancer is a glioma. In one embodiment, the glioma is a grade 1 glioma. In one embodiment, the glioma is a grade 2 glioma. In one embodiment, the glioma is a grade 3 glioma. In one embodiment, the glioma is a grade 4 glioma, which is also known as glioblastoma (GBM). In one embodiment the GBM is an IDH wild type GBM. In one embodiment, the GBM is an IDH mutant GBM.

In one embodiment, the human cell is a glioma cell. In one embodiment, the glioma cell is from a grade 1 glioma. In one embodiment, the glioma cell is from a grade 2 glioma. In one embodiment, the glioma cell is from a grade 3 glioma. In one embodiment, the glioma cell is from a grade 4 glioma, which is also known as glioblastoma (GBM). In one embodiment, the GBM cell is an IDH wild type GBM cell. In one embodiment, the GBM cell is an IDH mutant GBM cell.

In one embodiment, the human cell is from a human cell line. In one embodiment, the human cell line is a glioma cell line. In one embodiment, the glioma cell line is from a grade 1 glioma. In one embodiment, the glioma cell line is from a grade 2 glioma. In one embodiment, the glioma cell line is from a grade 3 glioma. In one embodiment, the glioma cell line is from a grade 4 glioma, which is also known as glioblastoma (GBM). In one embodiment, the GBM cell line is an IDH wild type GBM cell line. In one embodiment, the GBM cell line is an IDH mutant GBM cell line.

In one embodiment, the test compound comprises a polypeptide.

In one embodiment, the polypeptide comprises a portion of human Kvß2 or EAG2.

In one embodiment, the candidate therapeutic is compared to a control comprising the recombinant polypeptide as defined herein or the retro-inverso recombinant polypeptide as defined herein. For example, the ability of the candidate therapeutic to disrupt the interaction between EAG2 and Kvß2 may be compared to the corresponding activity of the control in disrupting interaction between EAG2 and Kvß2.

### Examples

### Identification of EAG2-Kv32 Potassium Channel Complex Reveals Glioblastoma Vulnerability to Designer Interference Peptide.

It has been discovered that voltage-gated potassium channel EAG2 regulates the growth⁴ and metastasis⁵ of medulloblastoma, the most common pediatric malignant brain tumor. As disclosed herein, chloride channel CLIC1 cooperates with EAG2 to regulate anion and cation flux and medulloblastoma growth⁶. In GBM, force-activated ion channel PIEZO1 confers mechanosensing ability to tumor cells. PIEZO1 promotes integrin-focal adhesion kinase signaling and tumor tissue stiffening. In turn, a stiffer microenvironment elevates PIEZO1 expression, creating a feedforward circuit to drive glioma aggression⁷. Therefore, ion channels, which mediate mechano-electrical-chemical signaling in tumor cells, are molecular dependencies in brain cancer.

It is shown that voltage-gated potassium channel ether a go-go (eag) and cytoplasmic potassium channel auxiliary subunit Hyperkinetic (Hk) regulate GBM growth in Drosophila. EAG2 (eag ortholog) and Kvß2 (Hk ortholog) synergistically regulate human GBM growth in mice. Kvß2 is required for plasma membrane localization of EAG2, which promotes GBM cell mitosis and GBM-neuron interaction. EAG2 and Kvß2 display physical interaction, a property conferred by a Kvß2 splice isoform 4 expressed in GBM cells. By engineering a series of cell-penetrable designer peptides, K90-114^{TAT} has been identified as a therapeutic peptide with potent anti-GBM efficacy in vitro and in vivo without noticeable toxicity on non-tumoral cells. Furthermore, K90-114^{TAT} displayed robust therapeutic efficacy against TMZ-resistant GBM. This not only identifies EAG2-Kvß2 potassium channel complex as a targetable vulnerability, but also establishes a designer interference peptide that disrupts this ion channel complex to treat GBM.

eag and Hk promote the growth of Drosophila melanogaster GBM.

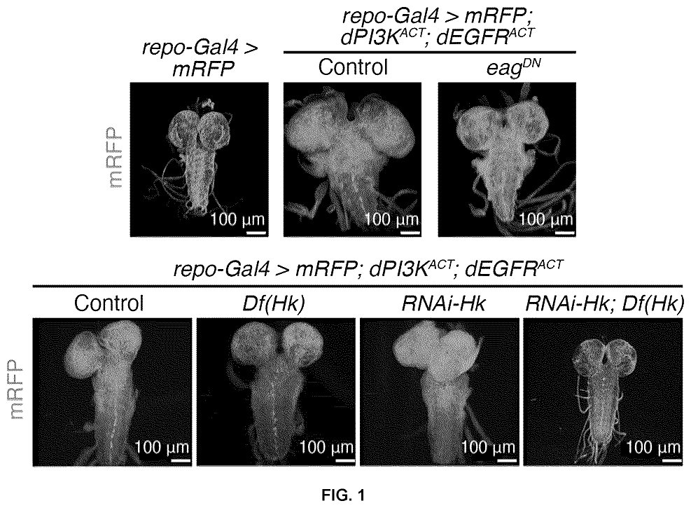

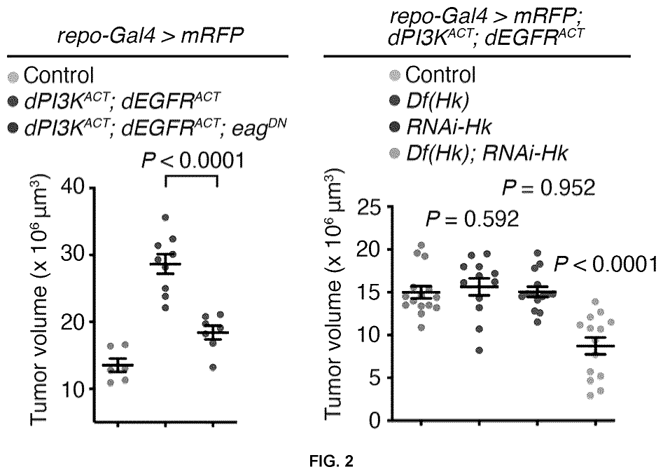

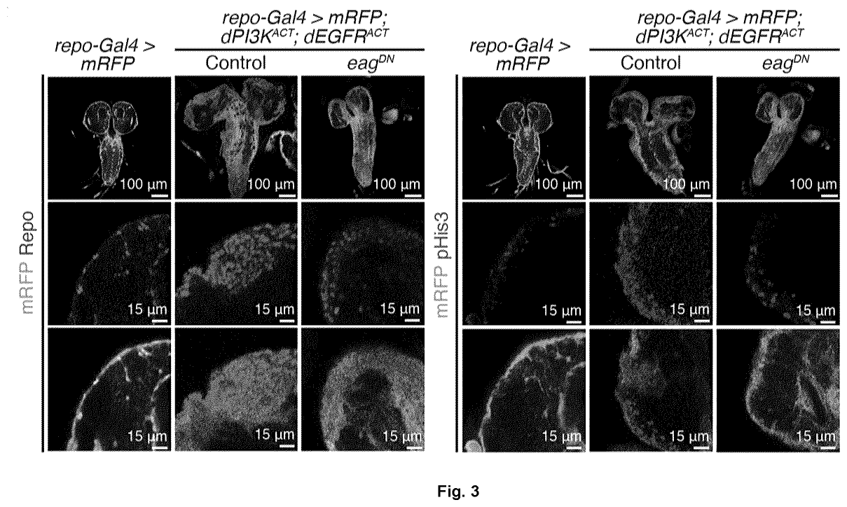

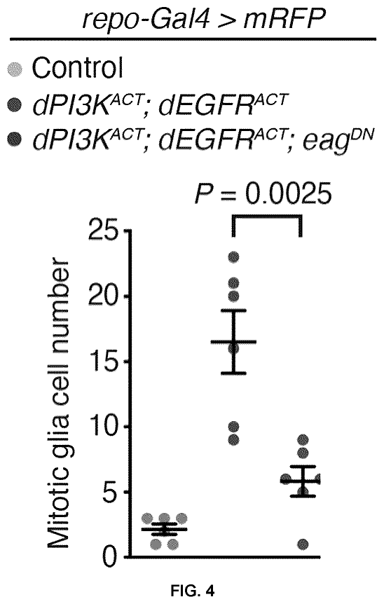

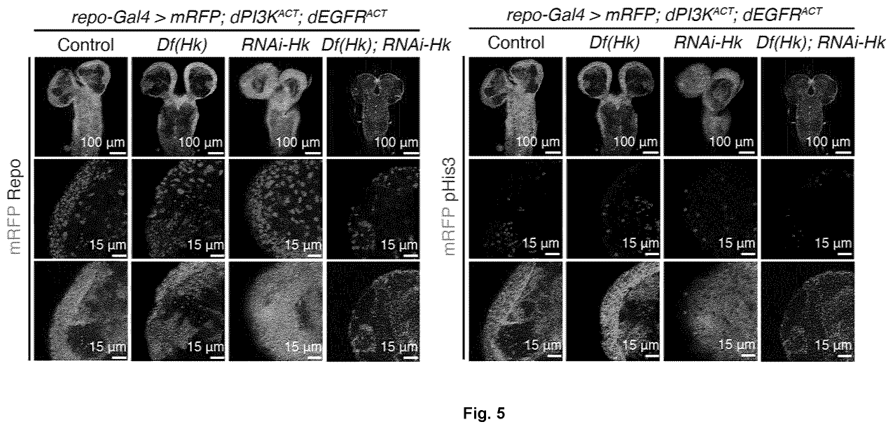

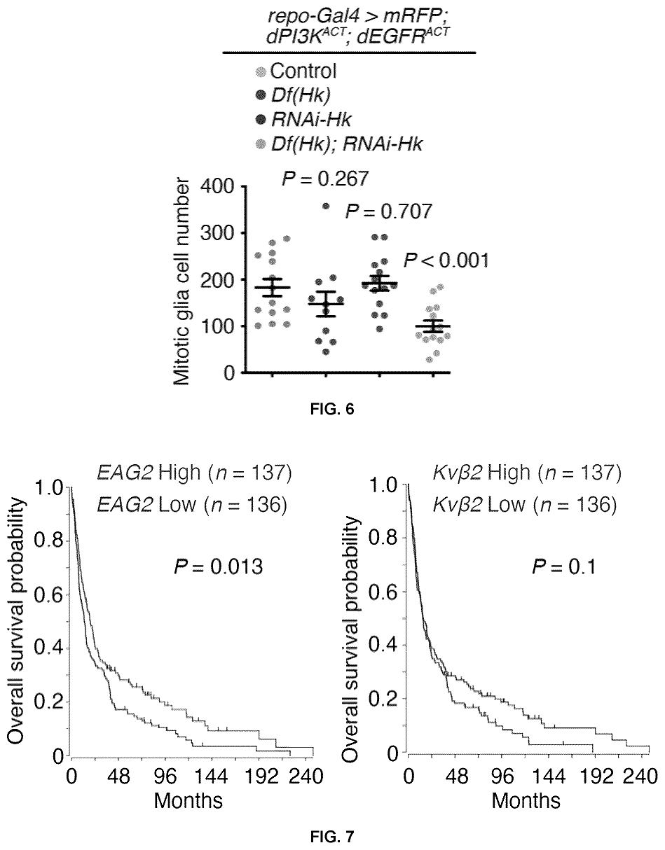

Activation of epidermal growth factor receptor (EGFR) and phosphatidylinositol-3 kinase (P13K) pathways are detected in over 40% of GBM patients⁸. Using glia-specific driver repo-Ga/4 to express constitutively active EGFR, P13K (dEGFR^{A}CT; dP/3K^{A}CT) and mRFP Drosophila melanogaster(fruit fly) GBM were generated that recapitulate characteristics of human GBM, including ectopic glia cell mitosis, increased total glia cell number, and enlarged brain tissues due to glial over-growth⁹. Tumor-specific expression of dominant negative eag markedly reduced tumor volume (FIG. 1 and FIG. 2). Similarly, tumor-specific knockdown of Hk decreased GBM growth (FIG. 1 and FIG. 2). Dominant negative eag or Hk knockdown reduced the numbers of mitotic glia cells and total glia cells (FIG. 3-6). These data demonstrate that eag and Hk promote tumor growth in a Drosophila model of GBM.

EAG2 and Kv32 promote the growth of human GBM

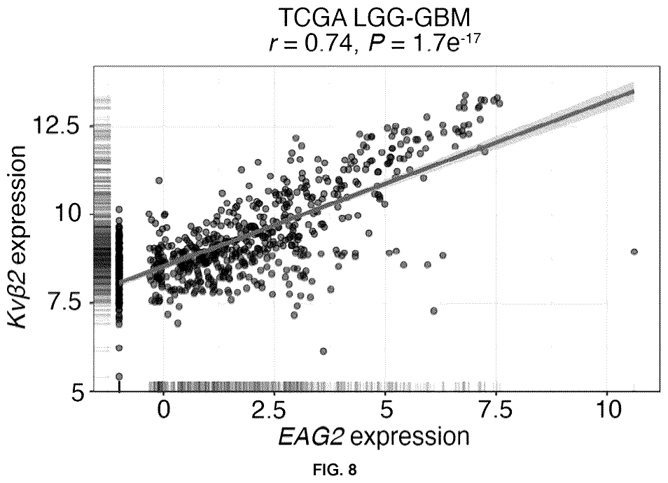

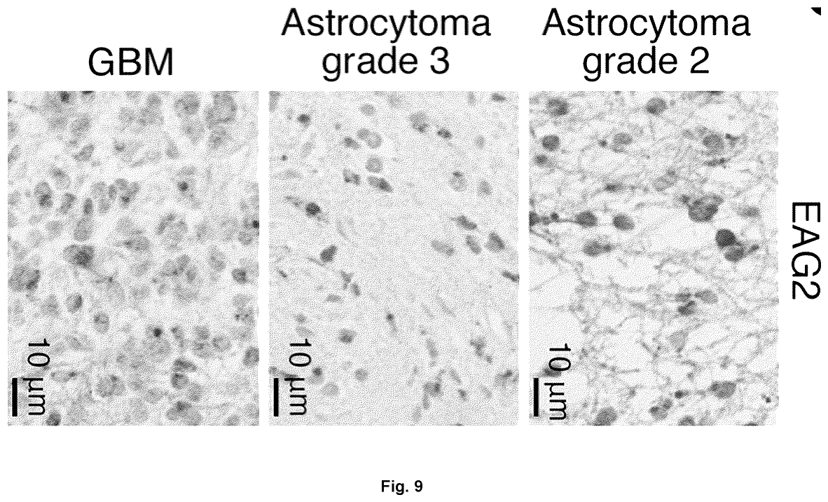

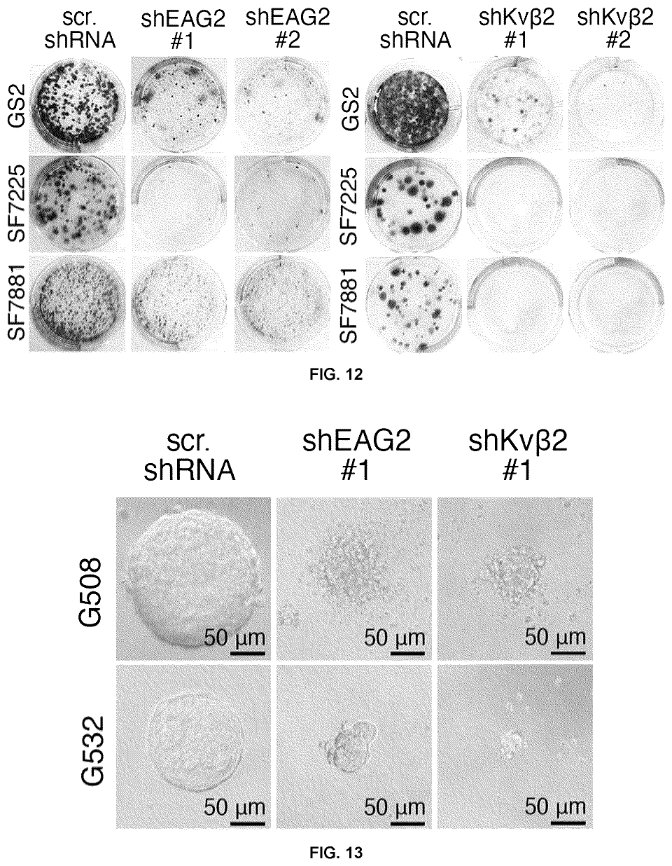

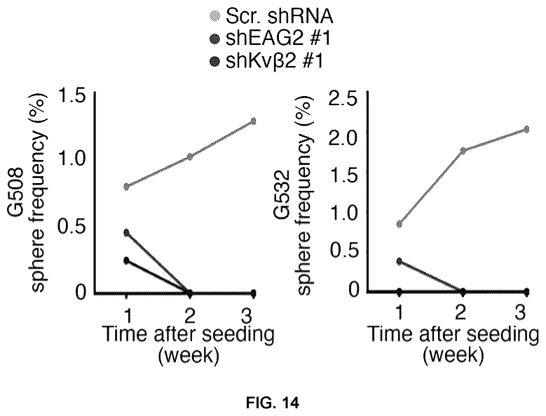

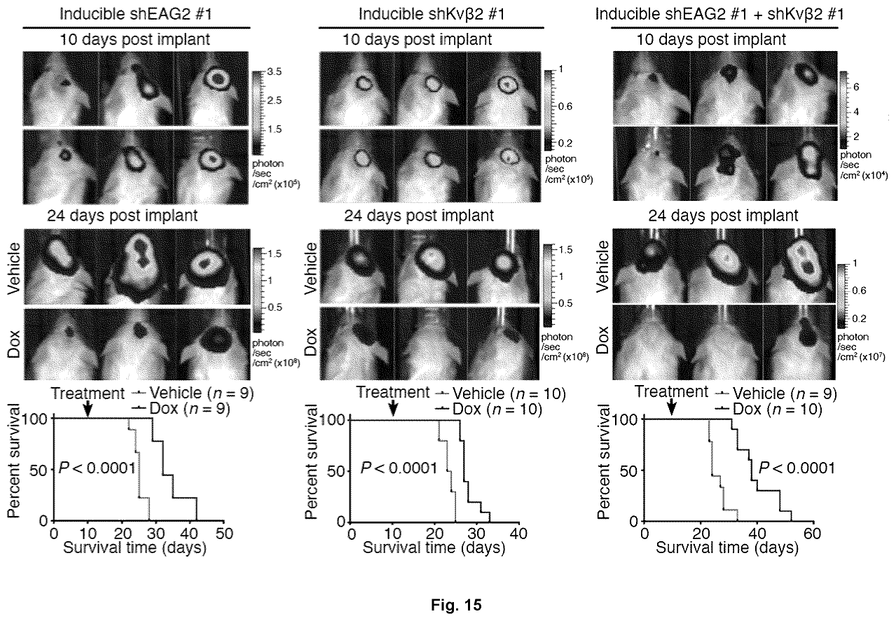

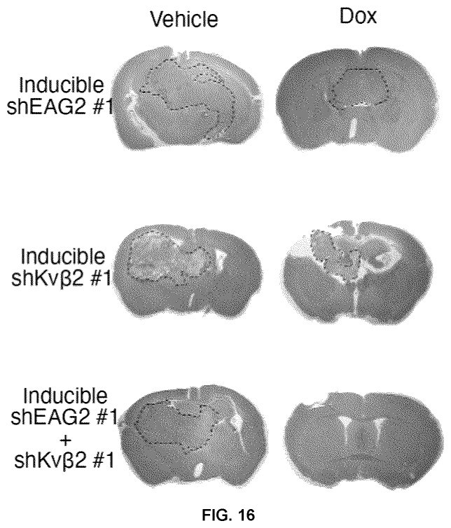

It was unknown whether the orthologs of Drosophila eag and Hk regulate human glioma malignancy. Accordingly, the association between the expression level of EAG1, EAG2 (eag orthologs), Kvß1, Kvß2 (Hk orthologs) and glioma patient survival has been investigated. High EAG2 or Kvß2 expression associates with shorter glioma patient survival (FIG. 7). EAG2 and Kvß2 expression displays strong positive correlation in human gliomas (FIG. 8). EAG2 expression is detected in human glioma tumor samples and patient-derived GBM cell lines (FIG. 9-11). Genetic knockdown of EAG2 or Kvß2 inhibits clonogenic growth and sphere forming abilities of GBM cells (FIG. 12-14). EAG2 or Kvß2 knockdown suppresses GBM growth and extends the survival of tumor-bearing mice in orthotopic xenograft models (FIG. 15 and FIG. 16). Importantly, combinatorial knockdown of EAG2 and Kvß2 cooperatively inhibits GBM growth (FIG. 15 and FIG. 16). These data establish EAG2 and Kvß2, which function cooperatively to promote tumor growth, as therapeutic vulnerabilities of GBM.

### EAG2 and Kvß2 Regulate GBM Cell Mitosis and GBM-Neuron Interaction

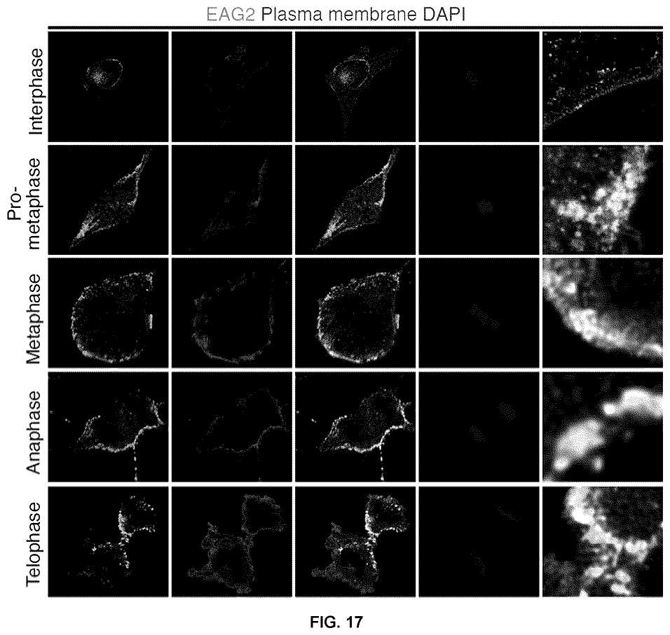

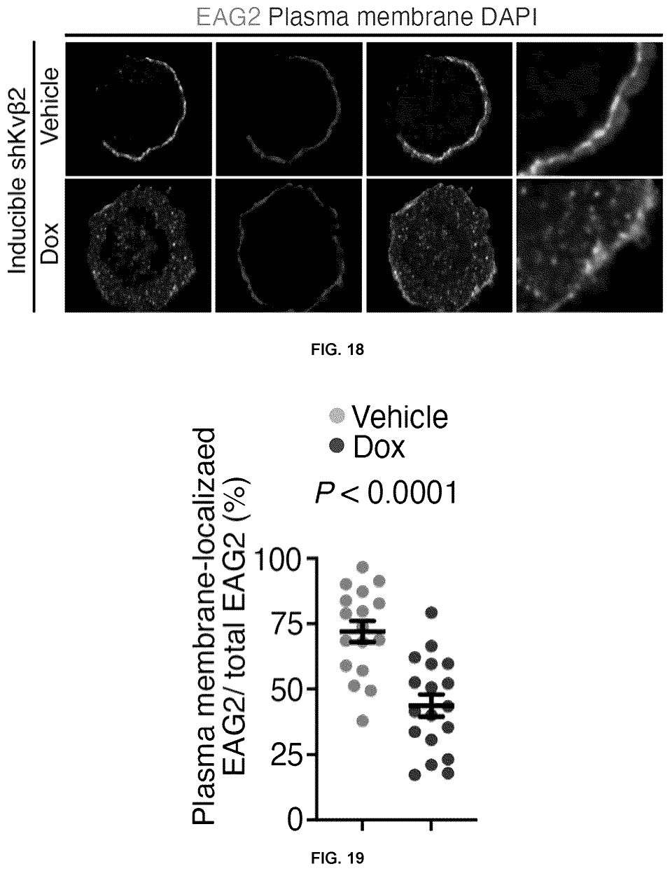

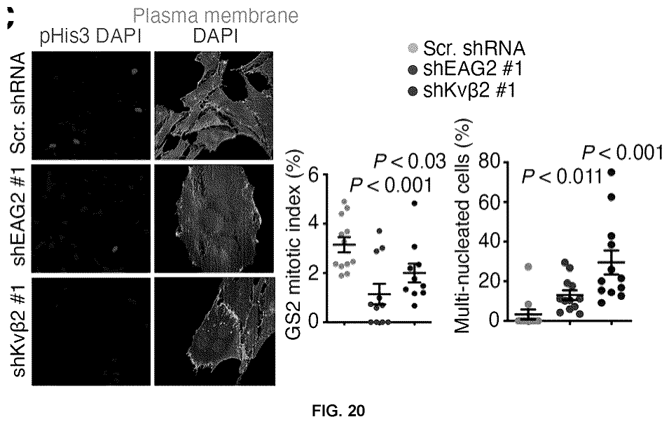

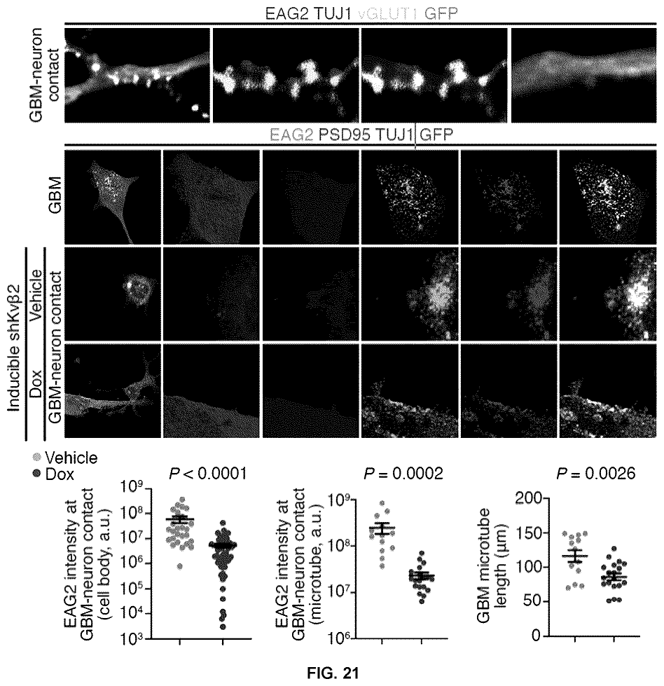

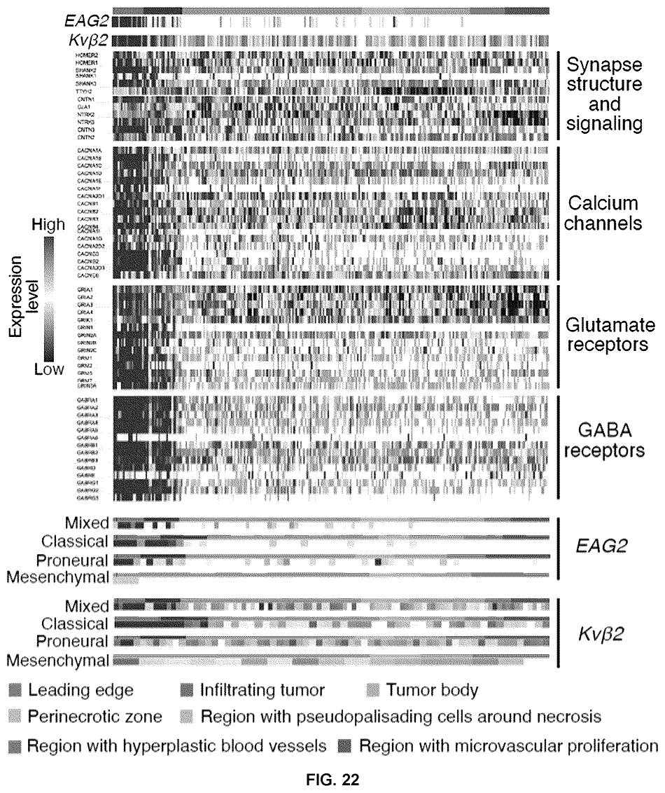

To determine the mechanism by which EAG2 and Kvß2 regulate GBM growth, the subcellular localization of EAG2 was studied during GBM cell cycle progression. While EAG2 localizes at intracellular compartments at interphase, it displays prominent plasma membrane localization during mitosis (FIG. 17). Kvß2 knockdown abrogates the mitosis-specific plasma membrane localization of EAG2 (FIGS. 18 and 19). EAG2 or Kvß2 knockdown decreases the mitotic index and led to multinucleation (an indicator of aberrant mitosis) of GBM cells (FIG. 20). Glioma cells develop synaptic connections with neurons, which induce neuronal activity-dependent depolarization of glioma cells to promote tumor proliferation and invasion¹⁰⁻¹². To determine whether EAG2 and Kvß2 regulate glioma-neuron interaction, human GBM cells are co-cultured with mouse hippocampal neurons. Hippocampal neurons extends axon to contact the body of GBM cells (FIG. 21). Interestingly, EAG2 and the post-synaptic marker PSD95 co-localize at GBM-neuron contact site (FIG. 21). Hippocampal neuron axon also contacts GBM cell processes, where EAG2 expression in tumor cells directly opposes the expression of pre-synaptic marker vGLUT1 in the axon (FIG. 21). Kvß2 knockdown reduces EAG2 and PSD95 distribution at GBM-neuron contact site (FIG. 21). High EAG2 or Kvβ62 expression associates with synaptic gene expression at the leading edge or infiltrating region of human GBM (FIG. 22). These data demonstrate that Kvß2 is required for EAG2 trafficking to the plasma membrane during mitosis of GBM cells, as well as EAG2 localization at the membrane contact sites between GBM cells and neurons.

Kvß2 isoform mediates physical interaction with EAG2 in GBM

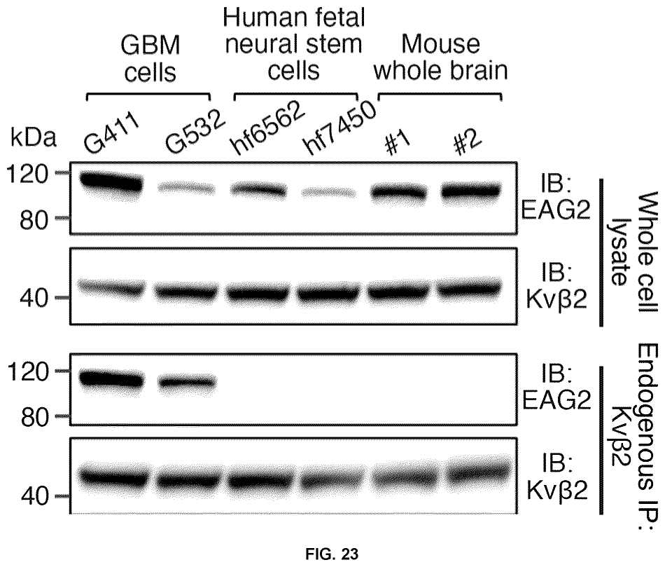

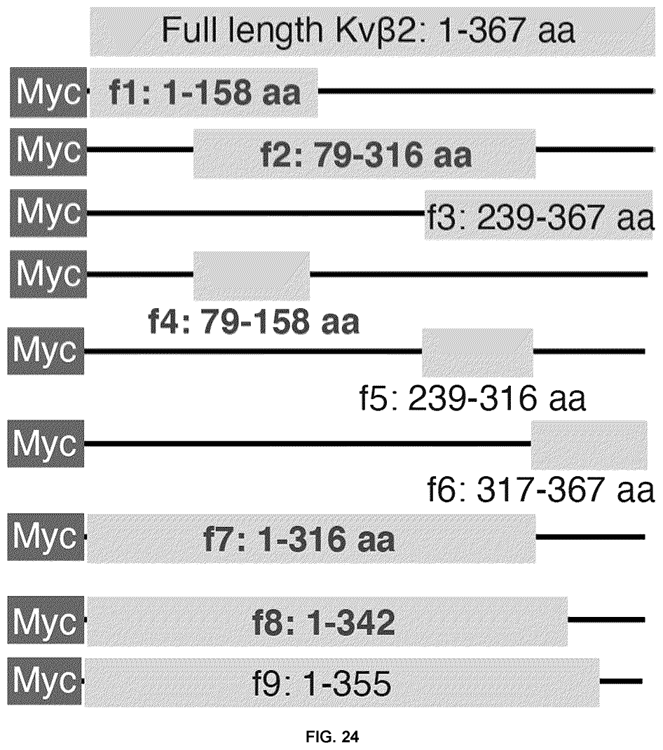

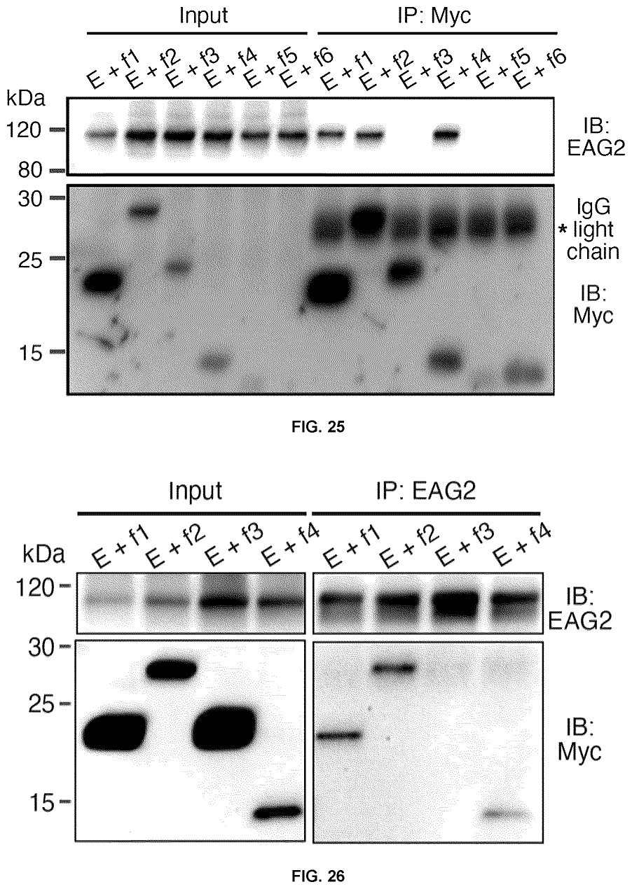

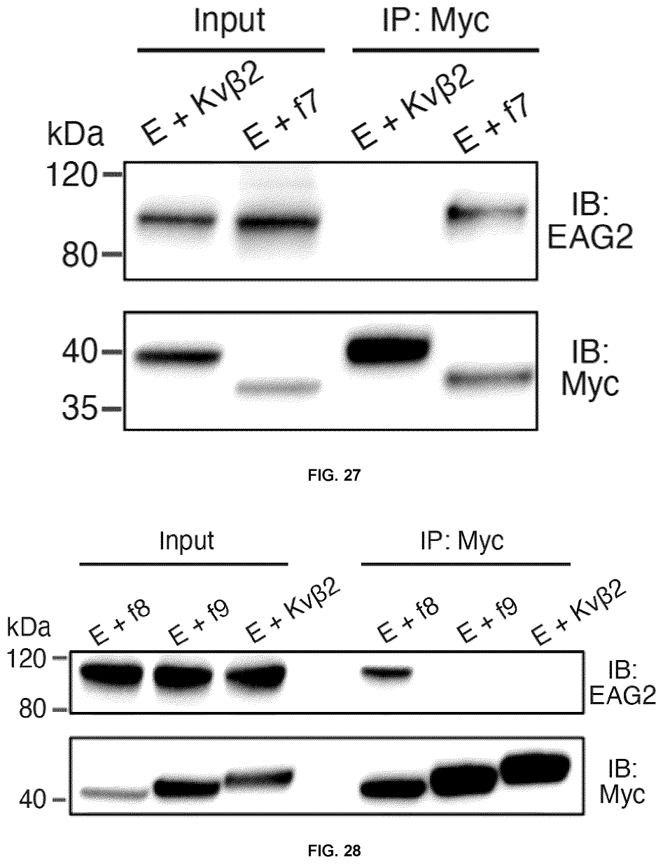

To determine whether EAG2 and Kvß2 display physical interaction, co-immunoprecipitation (co-IP) was performed using protein lysates from GBM cell lines, human fetal neural progenitor cell lines, and mouse whole brains. EAG2 interacted with Kvß2 only in GBM cells but not human fetal neural progenitor cells or mouse brains (FIG. 23). To elucidate Kvß2 amino acid sequence that mediates its interaction with EAG2, a series of Kvß2 fragment (f) mutants were generated (FIG. 24). Full length EAG2 and Kvß2 f mutants were expressed in HEK293T cells, followed by co-IP to determine the ability of each Kvß2 f mutant to interact with EAG2. First, Kvß2 was truncated into three overlapping f mutants: f1 (1-158 amino acid, aa), f2 (79-316 aa), and f3 (239-367 aa). f1 or f2 interacted with EAG2 (FIGS. 25 and 26), highlighting the importance of the overlapping amino acid sequence (79-158 aa) between f1 and f2. Indeed, f4 (79-158 aa) interacted with EAG2 (FIGS. 25 and 26). f3, or its two constituents f5 (239-316 aa) and f6 (317-367 aa), did not interact with EAG2 (FIGS. 25 and 26), demonstrating that Kvß2 C-terminus does not mediate interaction with EAG2. Next, the ability of f7 (1-316 aa), f8 (1-342 aa), and f9 (1-355 aa), which possess progressively shorter truncations at the C-terminus, to interact with EAG2 was determined. Intriguingly, f7, f8, but not f9, interacted with EAG2 (FIGS. 27 and 28), revealing amino acid 343-355 as an inhibitory sequence that prevents full length Kvß2 from interacting with EAG2.

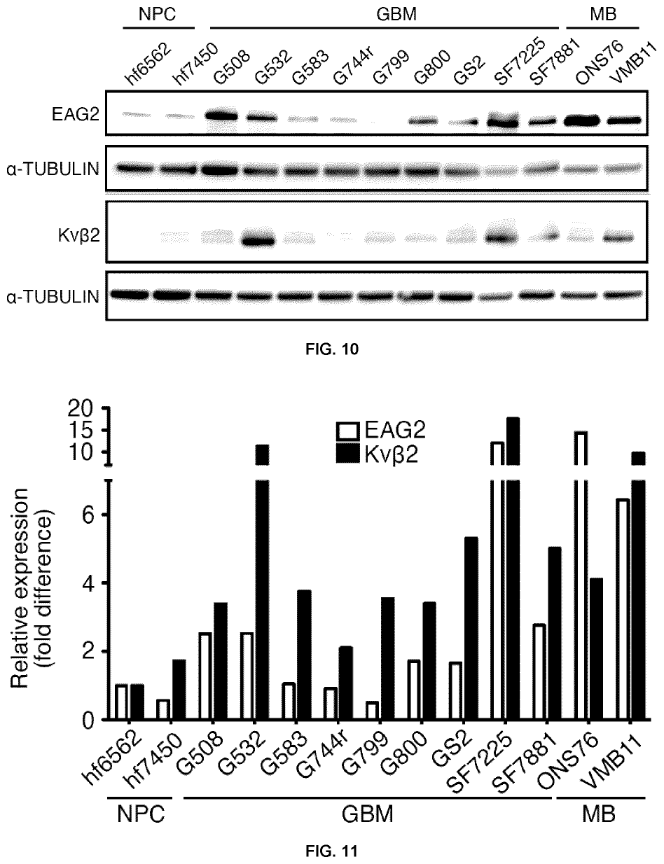

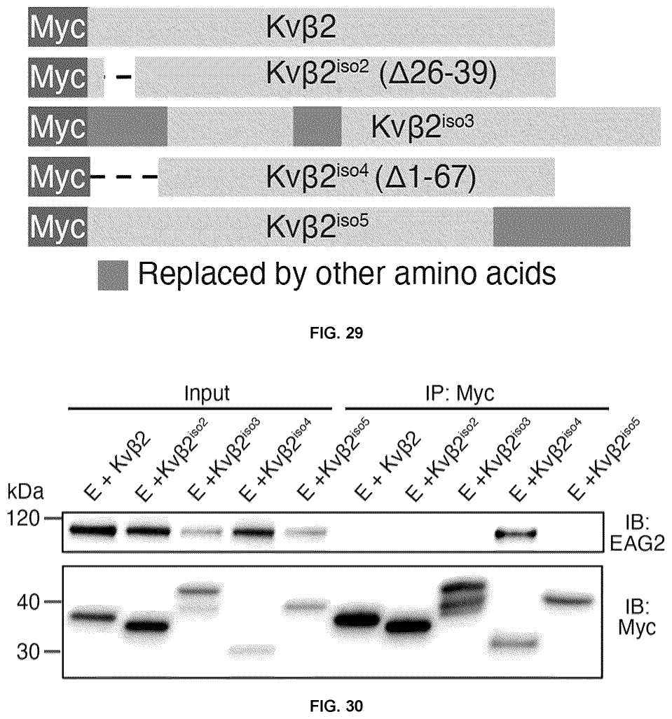

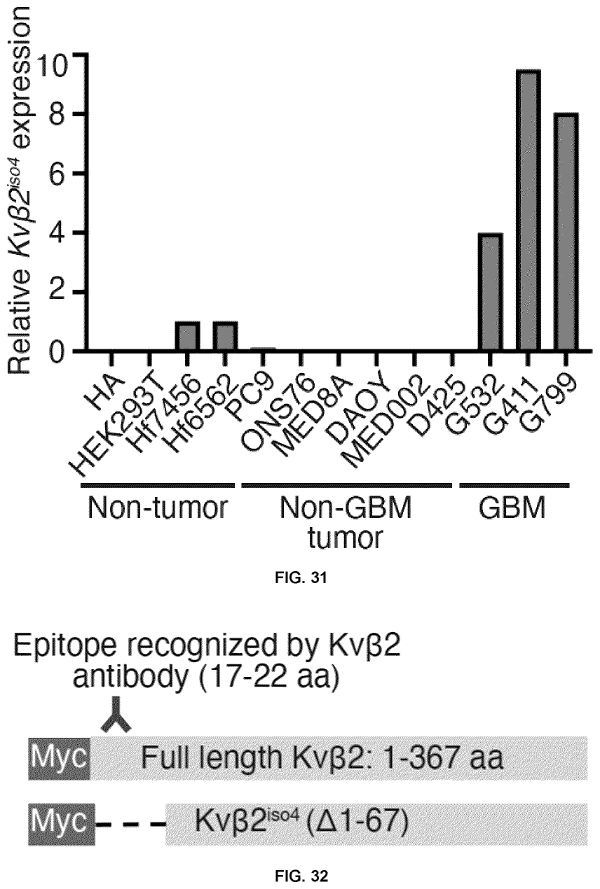

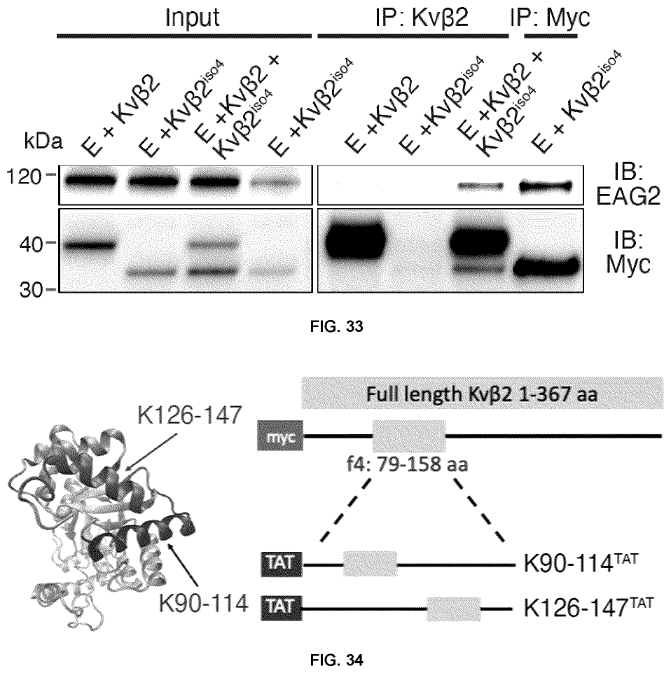

Although human fetal neural progenitor cells and mouse brain cells display EAG2 and Kvß2 expression, EAG2-Kvß2 interaction was not detected in these cell types (FIG. 10 and FIG. 23). Furthermore, transfection-mediated expression of full length EAG2 did not interact with Kvß2 in HEK293T cells (FIG. 27 and FIG. 28). These data suggest that GBM cells express a non-full length Kvß2 variant that possesses the ability to interact with EAG2. Consistent with this notion, GBM cell lines highly express Kvß2 isoform 4, which lacks amino acid 1-67 compared with full length Kvß2 (FIG. 29 and FIG. 30). Kvß2 isoform 4 interacted with EAG2 in HEK293T cells (FIG. 29 and FIG. 30). GBM cells, but not non-tumor or non-GBM tumoral cells, display high expression of Kvß2 isoform 4 (FIG. 31). Importantly, co-expressing Kvß2 isoform 4 and full length Kvß2 conferred EAG2-interacting ability to full length Kvß2 (FIG. 32, 33), revealing that the ability of amino acid 343-355 to inhibit EAG2-Kvß2 interaction requires amino acid 1-67. Taken together, these data identify Kvß2 isoform 4 expression in GBM cells, uncover amino acid 343-355 as an inhibitory sequence that prevents full length Kvß2 from interacting with EAG2, and reveal amino acid 79-158 that mediates EAG2-Kvß2 interaction.

### Designer Interference Peptide K90-114^{TAT} Disrupts EAG2-Kvß2 Interaction and Displays Therapeutic Efficacy in Treating GBM

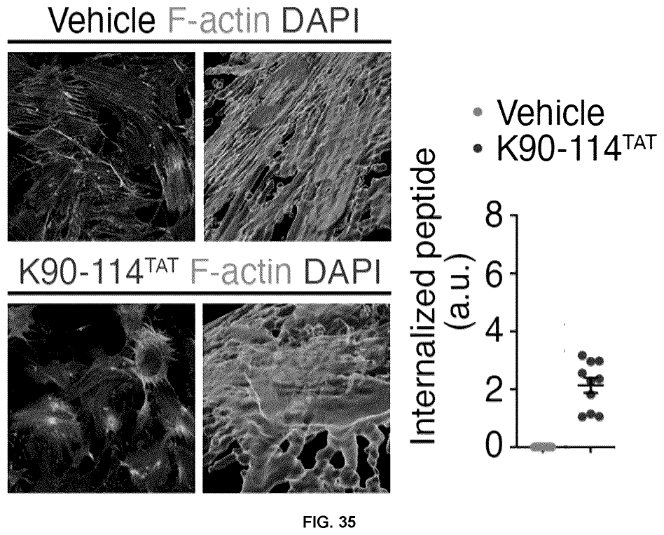

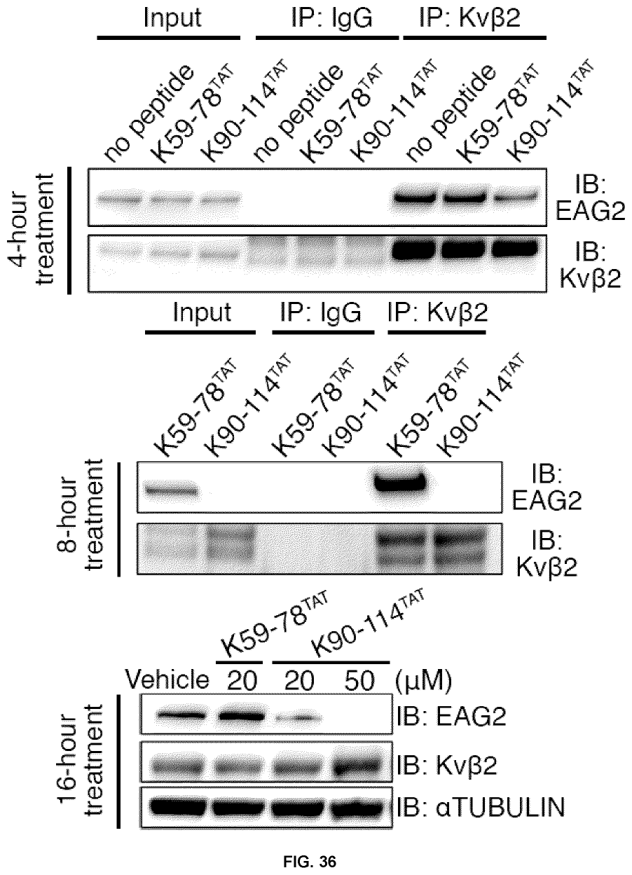

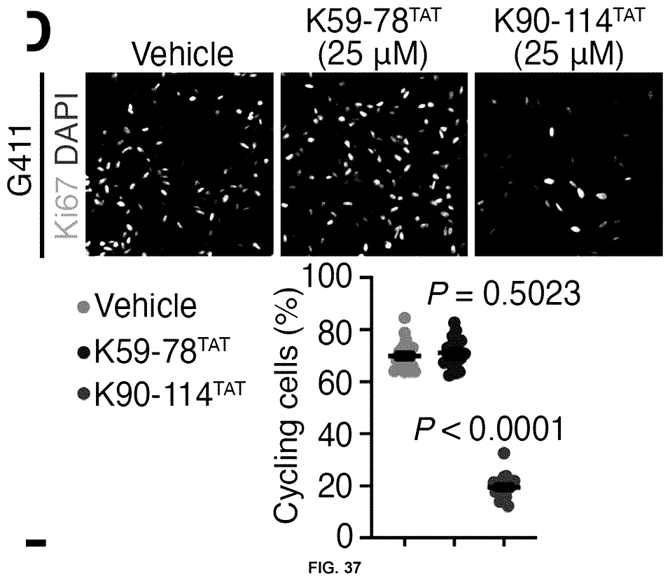

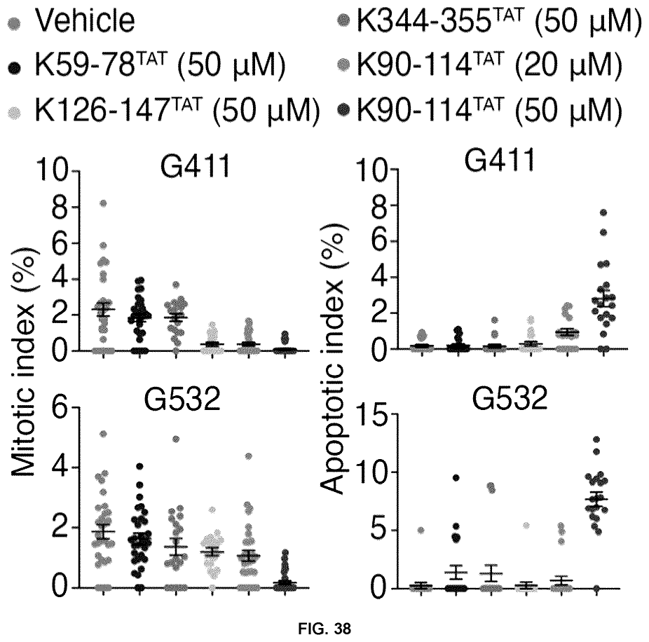

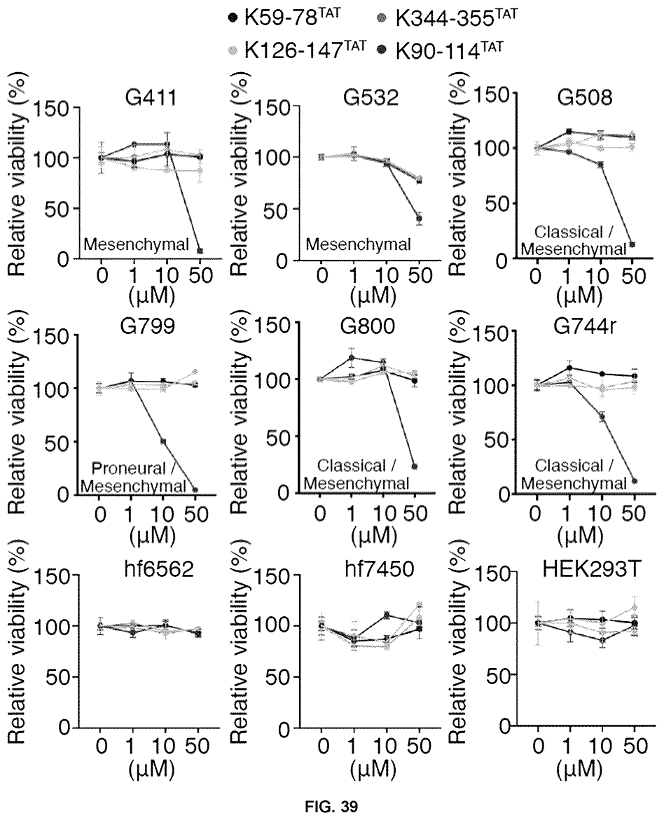

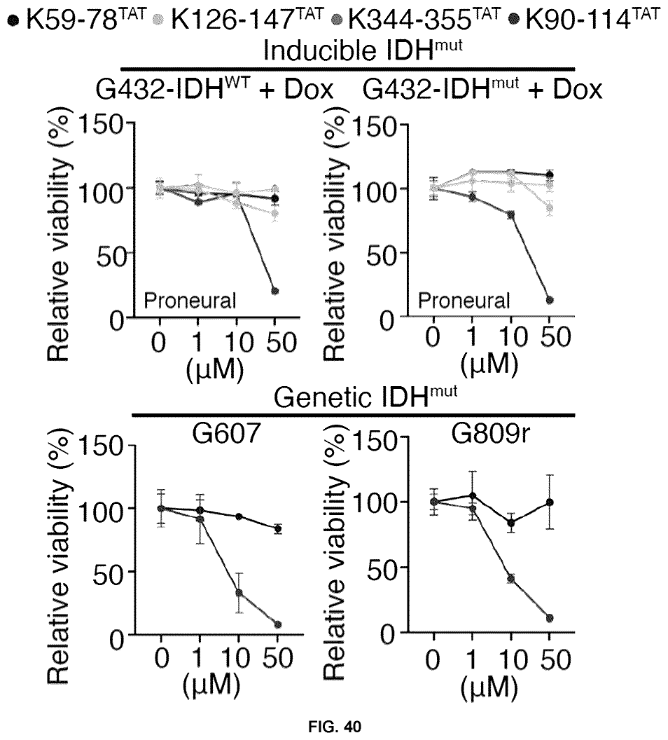

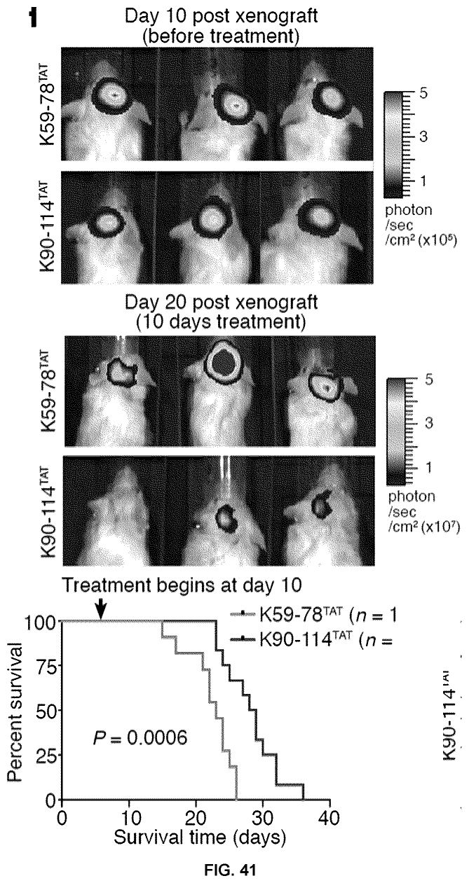

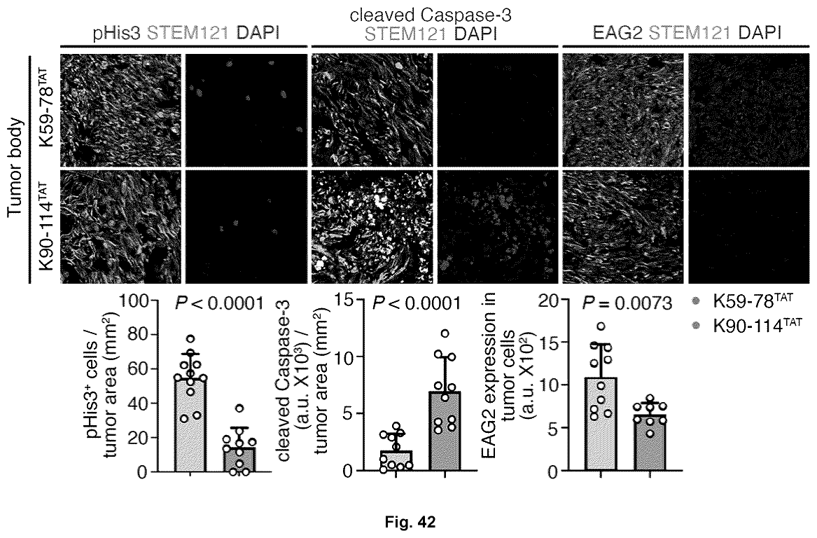

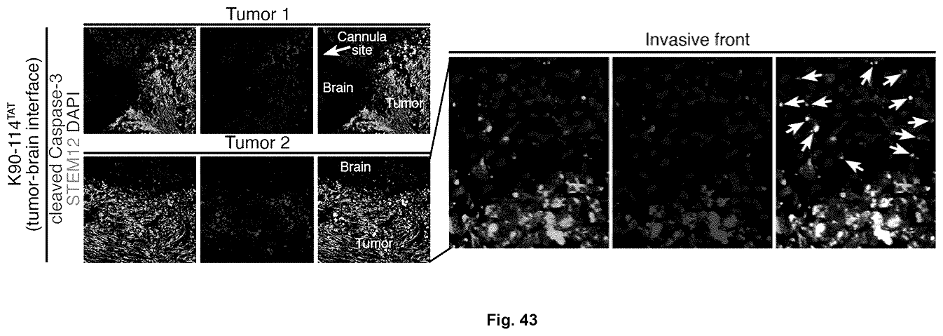

To design an approach to disrupt EAG2 and Kvß2 interaction, the f4 fragment of Kvß2, which includes amino acid 79-158, was explored. Intriguingly, this sequence contains 2 α-helices (amino acid 90-114 and amino acid 126-147) (FIG. 34). It was postulated that ectopic presence of these α-helices can competitively interfere with EAG2 and Kvß2 interaction. To test this hypothesis, cell-penetrable peptides were generated by adding a TAT sequence, which enhances cellular internalization, and a linker upstream of each α-helix, which were named K90-114^{TAT} or K126-147^{TAT} (FIG. 34 and FIG. 35). 4-hour treatment using K90-114^{TAT}, but not K59-78^{TAT}. reduced the interaction between endogenous EAG2 and Kvß2 in GBM cells (FIG. 36). 8-hour or 16-hour treatment using K90-114^{TAT}, but not K59-78^{TAT}, decreased EAG2 protein level (FIG. 36), suggesting that complexing with Kvß2 promotes EAG2 protein stability and/or expression. Next, TAT-modified peptides were generated for four α-helices within Kvß2 (K59-78^{TAT}, K90-114^{TAT} K126-147^{TAT}, K344-355^{TAT}) and their treatment effect on GBM cells was determined. Consistent with its ability to disrupt EAG2-Kvß2 interaction and decrease EAG2 protein level, K90-114^{TAT} but not other peptides, decreased mitosis, increased apoptosis, and reduced the overall viability of multiple GBM cell lines at concentrations that did not impact human fetal neural progenitor cells (hf6562, hf7450) or HEK293T cells (FIG. 37, FIG. 38, and FIG. 39). Furthermore, K90-114^{TAT}, but not other peptides, suppressed the growth of GBM cells with engineered expression of wild type or mutant IDH, as well as GBM cells with spontaneous IDH mutation (FIG. 40). Strikingly, cannula-mediated intratumoral delivery of K90-114^{TAT} suppressed GBM growth in an orthotopic xenograft model and extended the survival of tumor-bearing mice (FIG. 41). Consistent with in vitro data (FIG. 37, FIG. 38, and FIG. 39), K90-114^{TAT} treatment decreased mitosis and increased apoptosis of GBM cells, and reduced EAG2 expression in xenograft tumors (FIG. 42). Strikingly, K90-114^{TAT} treatment induced selective killing of GBM cells, which have invaded into tumor-adjacent brain parenchyma, but not the surrounding non-tumoral cells (FIG. 43). Therefore, we established K90-114^{TAT} as a designer interference peptide to disrupt EAG2-Kvß2 interaction with therapeutic efficacy in treating GBM.

### Designer Interference Peptide K90-114TAT as an Agent to Treat TMZ Resistant Gbm

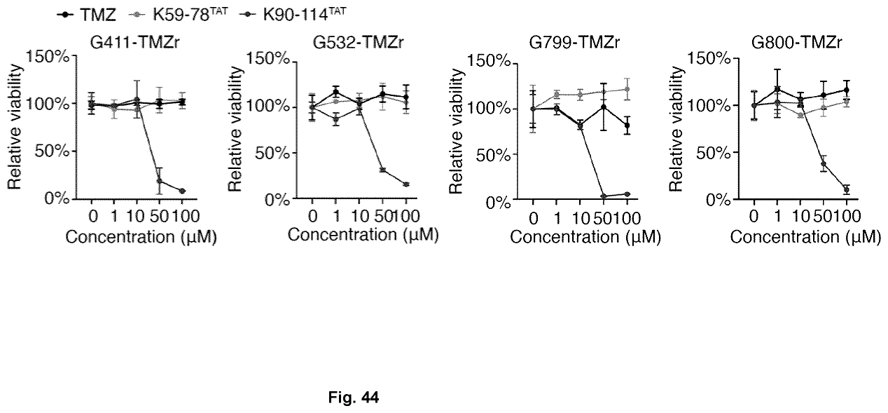

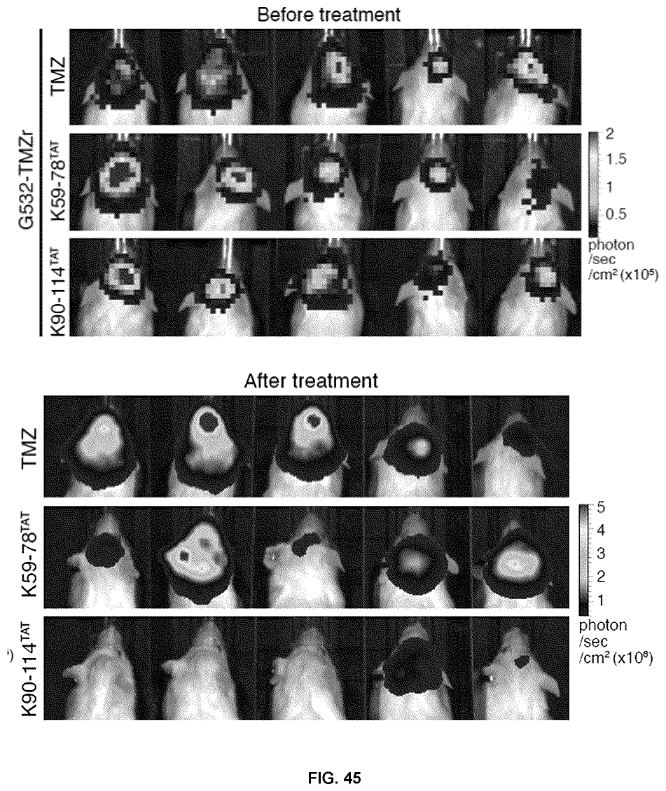

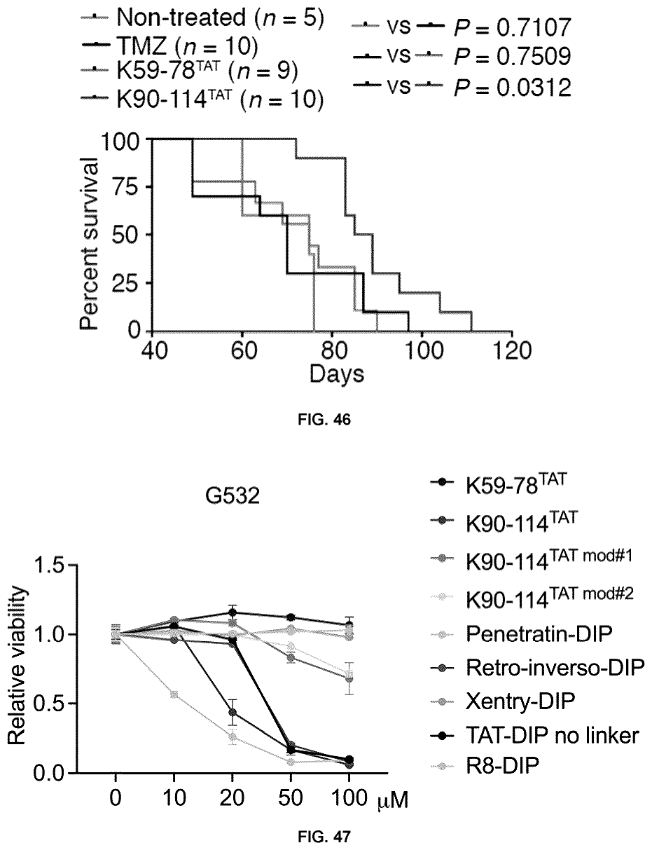

TMZ methylates adenine and guanine residues of DNA to form N³-methyladenine, N⁷-methylguanine, and O⁶-methylguanine, which leads to cell cycle arrest and apoptosis. The primary reasons for intrinsic and adaptive TMZ resistance include the activity of O⁶-methylguanine methyltransferase (MGMT), which repairs O⁶-methylation DNA damage induced by TMZ, alkylpurine-DNA-N-glycosylase (APNG), a base excision repair enzyme that repair N³-methyladenine and N⁷-methylguanine, or deficiency in DNA mismatch repair (MMR) in tumor cells¹. To investigate whether K90-114^{TAT} possesses therapeutic efficacy against TMZ-resistant GBM, GBM cell lines were generated by long-term treatment using increasing dosages of TMZ followed by selecting the resistant clones. Treating TMZ-resistant GBM cell lines with K90-114^{TAT}but not control peptide K59-78^{TAT} or TMZ, suppressed tumor cell growth (FIG. 44). Importantly, treating mice bearing TMZ-resistant GBM using K90-114^{TAT}, but not control peptide K59-78^{TAT} or TMZ, led to GBM regression in mice (FIG. 45) and survival extension (FIG. 46). These data establish K90-114^{TAT} as a novel agent for treating TMZ-resistant GBM.

### DISCUSSION

Since the discovery of concomitant therapy using TMZ and radiation, which improved GBM patient median survival relative to those treated with radiation alone from 12 to 15 months, all subsequent clinical trials failed to bring new molecularly targeted therapy into the clinics¹³. As a mainstay treatment for GBM, TMZ is a genotoxic mutagen, which can induce hypermutations that radically alter the genome to promote tumor heterogeneity and eventual therapy failures¹⁴. Furthermore, ˜50% GBM patients display upfront or acquired TMZ resistance, and combinatorial therapy to overcome TMZ resistance failed in clinical trials¹. Therefore, new molecular targets with GBM-selective mechanism of action are key to offer progress in this “untreatable disease”.

Ion channels are the third largest class of drug targets (after G protein-coupled receptors and kinases) for treating myriad human diseases. Membrane localization, tissue-specific expression, functional diversity, and known structure-activity relationships provide opportunities for ion channel drug discovery. However, unique challenges are present in developing ion channel modulators to treat brain cancer. First, ion channel functions are largely unknown in brain cancer. Second, small molecules display poor selectivity against members of the same ion channel family that has similar structural and functional domains. Third, the diverse ion channels essential for nervous system functions demands identification of cancer-specific mechanism amenable for therapeutic intervention. It is herein disclosed that EAG2 and Kvß2 co-regulate GBM growth in Drosophila and patient-derived xenograft models. It has been established that Kvß2 regulates plasma membrane localization of EAG2, which is required to promote GBM cell proliferation and communication with neurons. Kvß2 isoform 4 mediates EAG2-Kvß2 channel complex formation as a GBM-specific vulnerability. The identification of Kvß2 amino acid 79-158, which mediates the physical interaction between EAG2 and Kvß2, leads to rational design of a series of TAT-modified cell-penetrable peptides. Among these peptides, K90-114^{TAT} disrupts EAG2-Kvß2 interaction and displays robust therapeutic efficacy in treating both TMZ-sensitive and -resistant GBM. Therefore, the present disclosure encompasses identifying a new protein-protein interaction (PPI) as a GBM target, elucidating its mechanism of action, developing a first-in-class peptide for functional interference, and providing the first evidence of drugging PPI in ion channel complex to treat cancer.

PPI is often considered “undruggable” due to the absence of binding pocket in either of the individual proteins. Both ion channels and PPI, which require modulation of a large protein surface area to induce a therapeutic response, are recognized as ideal targets for peptide-based drugs. Nerinetide, which recently completed phase Ill trials for treating acute ischemic stroke, is composed of TAT-modified C terminus of NR2B9c, a 9-amino acid residue inhibitor of the interaction between PSD95 and NMDA (N-methyl-d-aspartate) receptors in neurons. TAT is engineered to deliver intravenously administered nerinetide across the blood-brain barrier. It is important to determine the pharmacokinetics, pharmacodynamics, potency, and potential side effect of peripherally administered K90-114^{TAT} in treating GBM. Wafer-mediated slow release of chemotherapeutic agent, such as carmustine (brand name GLIADEL), is used to treat GBM patients by placing the drug-containing wafer in the cavity after surgical removal of the tumor¹⁵. Intranasal delivery of the peptide hormone oxytocin has shown success in modulating social cognition and behaviors in humans¹⁶. Wafer-mediated slow release at tumor resection site or intranasal delivery of K90-114^{TAT} may also be considered as delivery routes for treating GBM.

K90-114^{TAT}, a first-in-class therapeutic compound that leverages the selectivity and low toxicity advantages of peptide, has been developed to target a cancer-specific ion channel mechanism to treat GBM. It is expected that medicinal chemistry to enhance K90-114^{TAT}bioavailability and stability will further increase its therapeutic use. Finally, it has been shown that EAG2 regulates the growth⁴ and metastasis⁵ of medulloblastoma. Identifying other cancer types, which utilize EAG2-Kvß2 potassium channel complex for malignant progression, will broaden the applicability of K90-114^{TAT} in oncology.

### Role of EAG2-Kvβ2 Complex in Regulating Electrical-Chemical Signaling Between GBM Cells and Neurons

Glioma cells and neurons form cancer-neuron synaptic connections. Electrical inputs from neurons signal to tumor cells to induce calcium signaling, membrane depolarization, GBM growth and invasion. Using genetic manipulations and designer interference peptide, the role of EAG2-Kvβ2 complex in regulating electrical-chemical signaling between GBM cells and neurons can be determined.

The electrical communications between GBM cells and neurons can be determined in vitro. GBM cells were generated with permanent expression of tdTomato, GCaMP6 (a genetically encoded calcium sensor), and doxycycline-inducible non-targeting shRNA, shRNA targeting EAG2, or shRNA targeting Kvβ2. It is expected that cortical neurons can be isolated from E18.5 (embryonic day 18.5) mouse embryos and GBM cell-neuron co-culture performed. Once GBM cells and neurons develop membrane-membrane contacts, live cell calcium imaging can be performed to detect calcium transients at GBM cell-neuron contact sites. First, the amplitude and frequency of calcium signals can be compared between GBM cells with or without neuronal contact. Then, doxycycline can be applied to cell culture medium to induce EAG2 or Kvβ2 knockdown. The amplitude and frequency of calcium signals can be compared between control GBM cells and GBM cells with EAG2 or Kvβ2 knockdown. Lastly, vehicle, control peptide K59-78^{TAT} and designer interference peptide K90_114^{TAT} can be applied to cell culture medium and calcium signals compared. These experiments can determine whether spontaneous neuronal activity induces calcium signaling in GBM cells, and whether such electrical communication depends on EAG2-Kvβ2 complex.

In parallel to calcium imaging, patch clamp recording can be performed. First, a single electrode can be used to record membrane potential dynamics of GBM cells with or without neuronal contact. Then, two-electrode recording can be performed, with one electrode electrically activating the neuron and the other electrode recording membrane potential of the GBM cell. Control GBM cells, and GBM cells can be compared with EAG2 or Kvβ2 knockdown, or GBM cells treated with vehicle, control peptide K59-78^{TAT}, or designer interference peptide K90-114^{TAT}These experiments can determine whether spontaneous neuronal activity- and evoked neuronal activity-induced electrical response of GBM cells depends on EAG2-Kvβ2 complex.

To determine electrical communications between GBM cells and neurons in vivo, GBM cells can be xenografted into CA1 region of hippocampus of immunocompromised mice. By feeding mice with doxycycline-containing food, inducible EAG2 or Kvβ2 knockdown can be achieved in tumor cells. Since Schaffer collaterals, which are axons of CA3 pyramidal cells in hippocampus, project to CA1 region, electrically stimulating Schaffer collaterals elicits calcium signaling in glioma cells in CA1. 2-3 weeks after xenograft, live GBM-containing tissue slice can be harvested. Two-electrode recording can be performed, in which one electrode electrically activates Schaffer collaterals and the other electrode records membrane potential of GBM cells located at CA1 region. Membrane potential dynamics in control GBM cells, and GBM cells can be compared with EAG2 or Kvβ2 knockdown. Vehicle, control peptide K59-78^{TAT}, or designer interference peptide K90-114^{TAT} can also be applied to the bath solution of the tumor-containing brain tissue slices, followed by two-electrode recording to compare membrane potential dynamics in GBM cells.

To uncover the biochemical signaling regulated by EAG2 and Kvβ2 in GBM cells, tdTomato⁺ GBM cells cultured with or without co-culturing with neurons can be isolated, or tdTomato⁺ GBM cells isolated from GBM-neuron co-culture with or without EAG2 or Kvβ2 knockdown. RNA-sequencing (RNA-seq) and proteomic profiling can be performed to determine neuronal signaling-induced transcriptomic and proteomic changes in vitro. tdTomato⁺ GBM cells of xenograft tumors with inducible EAG2 or Kvβ2 knockdown can be isolated and RNA-seq and proteomic profiling can be performed to define EAG2-Kvβ2-regulated genes and signaling pathways in vivo. Genes and signaling pathway that are commonly altered in vitro and in vivo can be identified, and functional manipulation can be performed to determine their roles in mediating the interactions between GBM cells and neurons.

Without wishing to be bound by any particular theory, it is expected that GBM cells and neurons develop electrical communications in vitro and in vivo. Spontaneous and/or evoked neuronal activity may induce depolarization of GBM cell membrane. It is expected that EAG2 or Kvβ2 knockdown impedes the repolarization phase of GBM cells after neuronal input, thereby resulting in defective GBM cell-neuron electrical coupling after repetitive neuronal inputs. It is expected that RNA-seq and proteomic profiling to reveal specific genes and signaling pathways that are regulated by EAG2 and Kvβ2 in a neuronal activity-dependent manner. Cancer-neuron synaptic coupling has only been recently identified, and the downstream signaling that mediates neuronal input-dependent tumor response is essentially unknown. These experiments will provide the foundation to define electrical-chemical signaling mechanisms in GBM.

### Efficacy of Designer Interference Peptide in Treating Post-Therapy Recurrent GBM

Patients with post-therapy (surgery, radiation, and TMZ treatment) GBM recurrence display particularly poor prognosis (median survival <6 months). The efficacy of using K90-114^{TAT} to treating recurrent GBM can be determined.

To determine the efficacy of K90-114^{TAT} in treating post-therapy GBM cells in vitro, clinically relevant TMZ and radiation treatment can be performed on GBM cell lines established from treatment-naïve tumors. GBM cells can be treated with 5 days of TMZ at 25 μM concurrently with 1 Gy per day of radiation, followed by additional 5 days of TMZ at 50 μM. Cells can be treated with TMZ for 1 hour per day, after which TMZ-containing medium can be replaced by fresh medium and cells will be exposed to 1 Gy radiation. After completion of this treatment scheme, GBM cells can be cultured until treatment-refractory cells are established. Such in vitro treatment enriches GBM cells with increased expression of stem cells genes and self-renewal capacity. Dose-dependent efficacy of K90-114^{TAT} in inducing cell death and decreasing proliferation of these treatment-refractory GBM cells can be determined.

To determine the efficacy of K90-114^{TAT} in treating post-therapy GBM in vivo, GBM cells can be orthotopically injected into immunocompromised mice and tumor growth monitored using non-invasive bioluminescence imaging. Once substantial tumor burdens are observed, tumor bulks can be surgically resected. Mice can be housed to recover for one week before receiving Stupp-like treatment, which includes radiation (2 Gy/day, 5 days) combined with TMZ (25 mg/kg, 5 days) followed by TMZ treatment alone (50 mg/kg, 5 days followed by 2 days without treatment for 4 weeks). Following Stupp-like treatment, tumor burden can be monitored by bioluminescence imaging. As GBM re-growth is detected, canula-guided, osmotic pump-mediated intratumoral delivery of vehicle, control peptide K59-78^{TAT}, or K90-114^{TAT} can be performed for 2 weeks. Immunostaining can be performed to compare tumor cell proliferation, apoptosis, and invasion. Mouse survival can be determined using Kaplan-Meier analysis. Multi-omics study can be performed, including bulk/single cell RNA-seq, proteomics, and metabolomics, to determine how peptide treatment impacts recurrent GBM to reveal additional tumor vulnerability induced by peptide treatment.

In addition to xenograft models, peptide efficacy can be studied using immunocompetent genetically engineered mouse models (GEMM) of GBM (GFAP-CreER; Pten^{flox/flox}; Tp53^{flox/flox} mice). Upon tamoxifen injection at P21 (postnatal day 21), GFAP-CreER; Pten^{flox/flox}; Tp53^{flox/flox}mice develop high grade glioma (including anaplastic astrocytoma and GBM). These tumors mimic human high-grade glioma by showing astrocytic phenotype, mitotic activity, cytological pleomorphism, and microvascular proliferation. Tumor incidence and growth can be monitored using 7T magnetic resonance imaging (MRI). After determining tumor locations, vehicle, control peptide K59-78^{TAT}, or K90-114^{TAT} treatment can be performed to determine peptide efficacy in treating these therapy-naïve gliomas. Furthermore, Stupp-like treatment can be performed and the therapeutic benefit of K90-114^{TAT} in treating post-therapy recurrent GBM can be determined.

Without wishing to be bound by any particular theory, it is expected that EAG2-Kvβ2 potassium channel complex regulates electrical-chemical signaling between GBM cells and neurons and designer interference peptide K90-114TAT can treat GBM and post-therapy recurrent GBM. It is expected that K90-114^{TAT} induces cell death and reduces proliferation in GBM cells and therapy-resistant GBM cells in vitro and post-therapy recurrent GBM in vivo. K90-114^{TAT} may selectively kill GBM cells without impacting on non-tumoral cells. Importantly, K90-114^{TAT} may ablate invasive GBM cells at infiltrating tumor region that cannot be removed by surgical resection. As a result, K90-114^{TAT} may suppress the growth of recurrent GBM to extend mouse survival.

EAG2-Kvβ2 potassium channel complex is a novel therapeutic target for GBM and related cancers, and peptides that disrupt the interaction of EAG2 and Kvβ2 are promising therapeutic candidates. It can be expected that other molecules that disrupt the interaction of EAG2 and Kvβ2 will be useful in this regard, and these may include therapeutic polypeptides including or encompassing at least portions of the regions identified herein as being important for EAG2-Kvβ2 interaction.

### Cell Viability Following Peptide Treatment

GBM532 cells were exposed to increasing concentrations of various peptide treatments. FIG. 47 depicts these results. The peptide K59-78^{TAT}was used as a negative control. Good cell-killing activity was observed for K90-114TAT, Penetratin-DIP, Retro-inverso-DIP, and TAT-DIP with no linker.

### K90-114^{TAT} Treatment is Effective Against TMZ-Resistant GBM

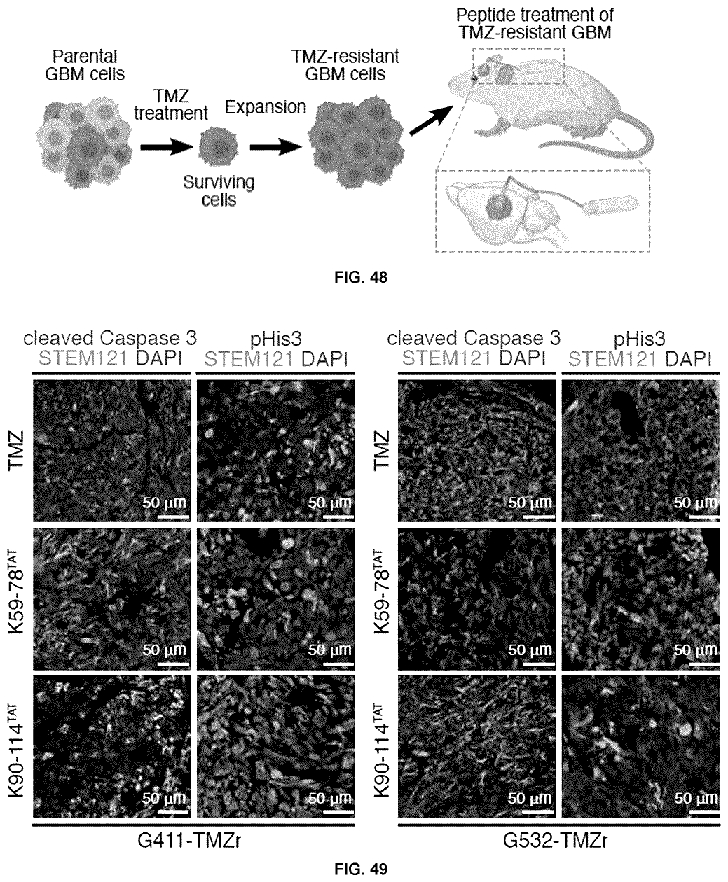

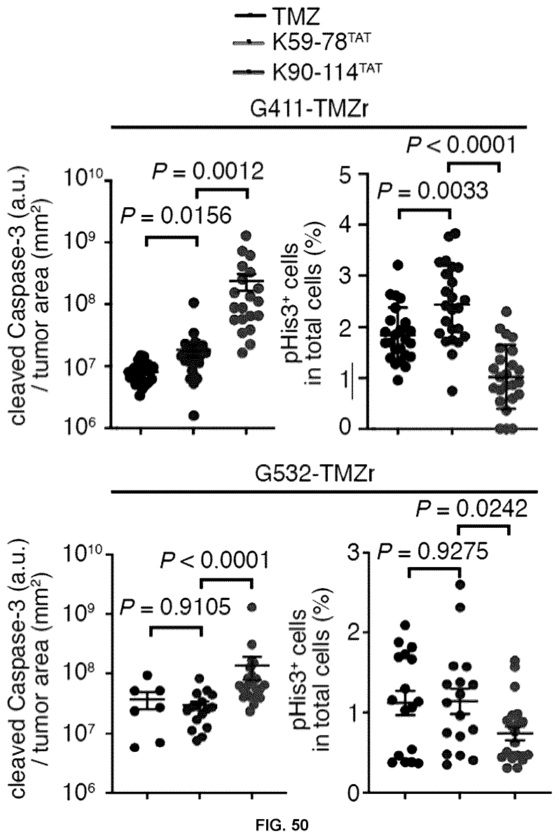

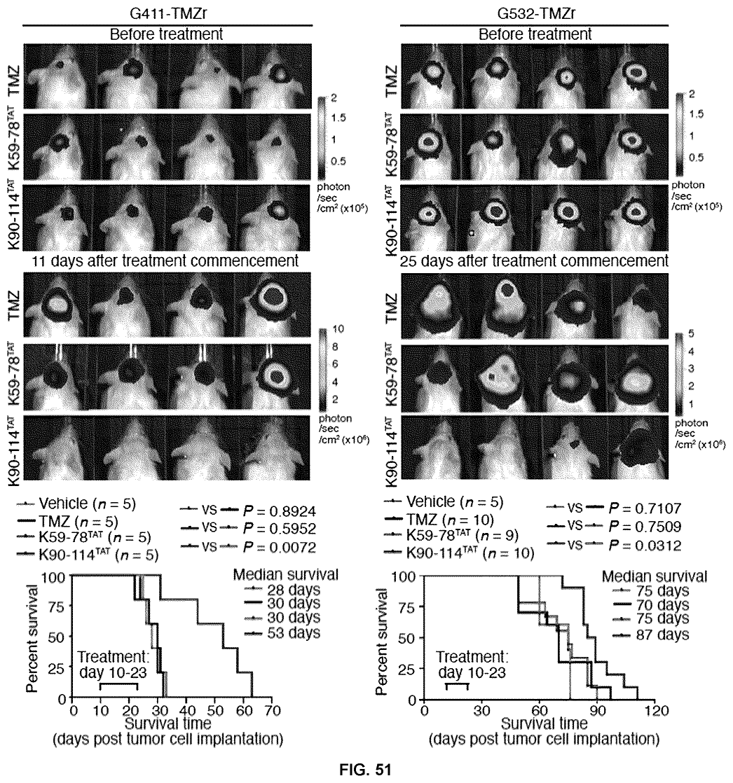

Since TMZ is a cornerstone of GBM therapy, TMZ resistance underlies tumor recurrence and the eventual treatment failure. To establish designer peptide K90-114^{TAT} as a therapeutic option for TMZ-resistant GBM patients, GBM cells were treated with increasing concentrations of TMZ (up to 400 μM), surviving cells were selected, TMZ-resistant cell lines were established, and orthotopic xenografts were performed (see FIG. 48 for a schematic). G411-TMZr and G532-TMZr xenograft models were studied to determine the therapeutic efficacy of a two-week peptide treatment regime on rapid- and slow-growing tumors, respectively. After TMZ-resistant GBM tumors displayed substantial in vivo growth, mice were treated with TMZ, K59-78^{TAT}(negative control), or K90-114^{TAT}. Treating mice with K90-114^{TAT}, but not K59-78^{TAT} or TMZ, decreased tumor cell proliferation, increased tumor cell apoptosis (FIGS. 49 and 50), resulting in significantly reduced tumor burden and extension of mouse survival (FIG. 51). In comparison to the marked therapeutic benefit seen in mice bearing G411-TMZr tumors, the modest, yet significant, benefit seen in K90-114^{TAT}-treated G532-TMZr-bearing mice was likely due to that the two-week treatment period constituted a relatively shorter therapy administration window in the overall longer tumor growth and mouse survival time (FIG. 51).

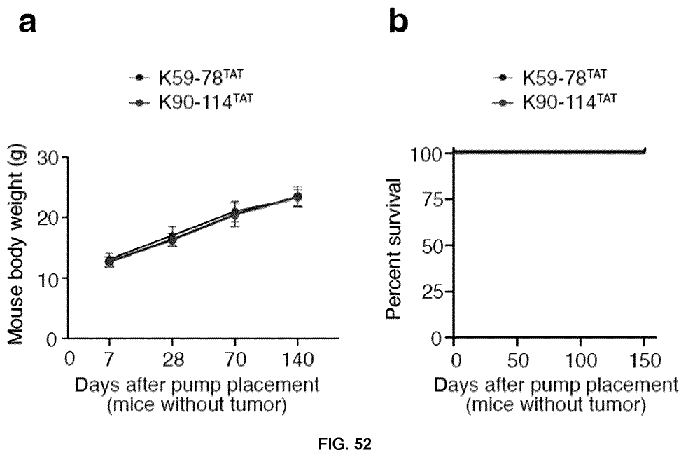

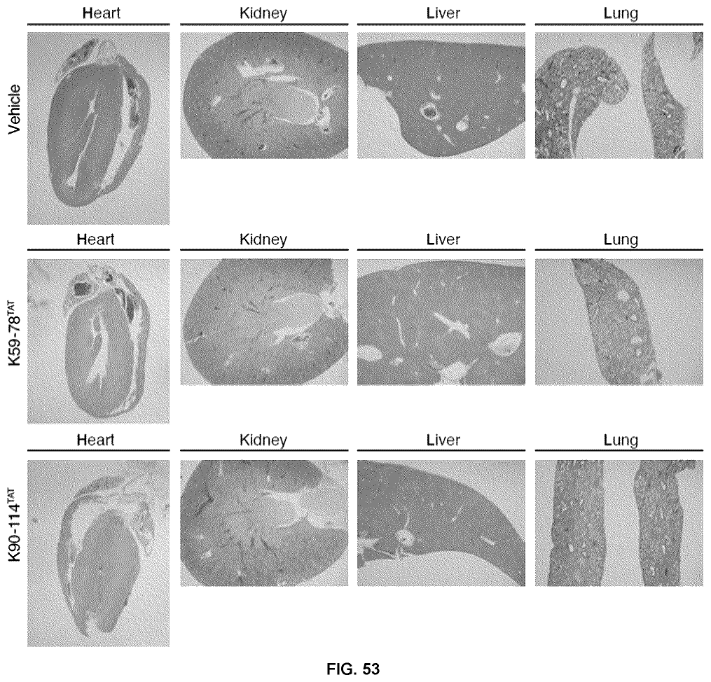

To further determine the impact of peptide treatment on mouse physiology, non-tumor-bearing mice were treated with K59-78^{TAT} or K90-114^{TAT}. K59-78^{TAT} and K90-114^{TAT}_treated mice displayed comparable body weight and survival (FIG. 52, Panels a & b). Inspections of internal organs (heart, kidney, liver, lung) did not reveal pathological features in mice treated with either peptide (FIG. 53).

### REFERENCES

1 Lee, S. Y. Temozolomide resistance in glioblastoma multiforme. Genes Dis 3, 198-210, doi:10.1016/j.gendis.2016.04.007 (2016).
2 Overington, J. P., AI-Lazikani, B. & Hopkins, A. L. How many drug targets are there? Nat Rev Drug Discov 5, 993-996, doi:10.1038/nrd2199 (2006).
3 Fan, J. J. & Huang, X. Ion Channels in Cancer: Orchestrators of Electrical Signaling and Cellular Crosstalk. Rev Physiol Biochem Pharmacol, doi:10.1007/112_2020_48 (2020).
4 Huang, X. et al. Voltage-gated potassium channel EAG2 controls mitotic entry and tumor growth in medulloblastoma via regulating cell volume dynamics. Genes & development 26, 1780-1796, doi:10.1101/gad.193789.112 (2012).
5 Huang, X. et al. EAG2 potassium channel with evolutionarily conserved function as a brain tumor target. Nature neuroscience 18, 1236-1246, doi:10.1038/nn.4088 (2015).
6 Francisco, M. A. et al. Chloride intracellular channel 1 cooperates with potassium channel EAG2 to promote medulloblastoma growth. J Exp Med 217, doi:10.1084/jem.20190971 (2020).
7 Chen, X. et al. A Feedforward Mechanism Mediated by Mechanosensitive Ion Channel PIEZO1 and Tissue Mechanics Promotes Glioma Aggression. Neuron, doi:10.1016/j.neuron.2018.09.046 (2018).
8 Agnihotri, S. et al. Glioblastoma, a brief review of history, molecular genetics, animal models and novel therapeutic strategies. Arch Immunol Ther Exp (Warsz) 61, 25-41, doi:10.1007/s00005-012-0203-0 (2013).
9 Read, R. D., Cavenee, W. K., Furnari, F. B. & Thomas, J. B. A drosophila model for EGFR-Ras and P13K-dependent human glioma. PLoS genetics 5, e1000374, doi:10.1371/journal.pgen.1000374 (2009).
10 Venkataramani, V. et al. Glutamatergic synaptic input to glioma cells drives brain tumour progression. Nature 573, 532-538, doi:10.1038/s41586-019-1564-x (2019).
11 Venkatesh, H. S. et al. Electrical and synaptic integration of glioma into neural circuits. Nature 573, 539-545, doi:10.1038/s41586-019-1563-y (2019).
12 Zeng, Q. et al. Synaptic proximity enables NMDAR signalling to promote brain metastasis. Nature 573, 526-531, doi:10.1038/s41586-019-1576-6 (2019).
13 Lukas, R. V. et al. Newly Diagnosed Glioblastoma: A Review on Clinical Management. Oncology (Williston Park) 33, 91-100 (2019).
14 Choi, S. et al. Temozolomide-associated hypermutation in gliomas. Neuro Oncol 20, 1300-1309, doi:10.1093/neuonc/noy016 (2018).
15 Perry, J., Chambers, A., Spithoff, K. & Laperriere, N. Gliadel wafers in the treatment of malignant glioma: a systematic review. Curr Oncol 14, 189-194, doi:10.3747/co.2007.147 (2007).
16 Quintana, D. S. et al. Advances in the field of intranasal oxytocin research: lessons learned and future directions for clinical research. Mol Psychiatry 26, 80-91, doi:10.1038/s41380-020-00864-7 (2021).

In the preceding description, for purposes of explanation, numerous details are set forth in order to provide a thorough understanding of the embodiments. However, it will be apparent to one skilled in the art that these specific details are not required.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All references referred to herein are incorporated by reference in their respective entireties.

What is claimed is:

1. A recombinant polypeptide comprising:
a) a polypeptide for preventing or reducing interaction between EAG2 and Kvß2, and
b) a cell-penetrating peptide.

2. A recombinant polypeptide comprising:
a) a polypeptide for preventing or reducing interaction between EAG2 and Kvß2 comprising:
i) at least 5 contiguous amino acids from a region of human Kvß2 (SEQ ID NO: 26) selected from the group consisting of:
amino acids 1 to 67 thereof,
amino acids 90 to 114 thereof, and
amino acids 343 to 355 thereof, or
ii) a polypeptide that is at least 70% identical to i); and
b) a cell-penetrating peptide.

3. A recombinant polypeptide comprising:
a) a polypeptide for preventing or reducing interaction between EAG2 and Kvß2 comprising:
i) a contiguous portion of human Kvß2 (SEQ ID NO: 26) encompassing at least amino acids 90 to 114 thereof, or
ii) a polypeptide that is at least 70% identical to i); and
b) a cell-penetrating peptide.

4. The recombinant polypeptide of claim 2 or 3, wherein a) the polypeptide for preventing or reducing interaction between EAG2 and Kvß2 is as defined in a) ii).

5. The recombinant polypeptide of claim 4, wherein the polypeptide for preventing or reducing interaction between EAG2 and Kvß2 is at least 80% identical to the polypeptide of i).

6. The recombinant polypeptide of claim 4 wherein e polypeptide for preventing or reducing interaction between EAG2 and Kvß2 is at least 90% identical to the polypeptide of i).

7. The recombinant polypeptide of claim 2 or 3, wherein a) the polypeptide for preventing or reducing interaction between EAG2 and Kvß2 is as defined in a) i).

8. The recombinant polypeptide of claim 2 or 3, wherein the polypeptide for preventing or reducing interaction between EAG2 and Kvß2 comprises amino acids 90 to 114 of human Kvß2 (SEQ ID NO: 1).

9. The recombinant polypeptide of claim 2 or 3, wherein the polypeptide for preventing or reducing interaction between EAG2 and Kvß2 consists of amino acids 90 to 114 of human Kvß2 (SEQ ID NO: 1).

10. The recombinant polypeptide of any one of claims 1 to 9, wherein the cell-penetrating peptide is spaced apart from the polypeptide for preventing or reducing interaction between EAG2 and Kvß2 by an amino acid linker.

11. The recombinant polypeptide of claim 10, wherein the amino acid linker comprises amino acids GSGSGS (SEQ ID NO: 16).

12. The recombinant polypeptide of any one of claims 1 to 11, wherein the cell-penetrating peptide is selected from the group consisting of:
HIV TAT (SEQ ID NO: 5),
Circularizable HIV TAT (SEQ ID NO: 6),
HA-TAT (SEQ ID NO: 7),
Penetratin (SEQ ID NO: 8),
Circularizable Penetratin (SEQ ID NO: 9),
Transportan (SEQ ID NO: 10), and
Circularizable Transportan (SEQ ID NO: 11).

13. The recombinant polypeptide of any one of claims 1 to 12, wherein the cell-penetrating peptide is positioned N-terminally with respect to the polypeptide for preventing or reducing interaction between EAG2 and Kvß2.

14. The recombinant polypeptide of any one of claims 1 to 11, wherein the cell-penetrating peptide comprises HIV TAT (SEQ ID NO: 5), which is positioned N-terminally with respect to the polypeptide for preventing or reducing interaction between EAG2 and Kvß2.

15. A recombinant polypeptide, which comprises amino acids having SEQ ID NO: 17.

16. A recombinant polypeptide, which comprises amino acids having SEQ ID NO: 18.

17. A retro-inverso polypeptide comprising D-amino acids in an order reverse to that of amino acids of the recombinant polypeptide as defined in any one of claims 1 to 16.

18. The retro-inverso polypeptide of claim 17, wherein the D-amino acids comprise the sequence of amino acid positions 1 to 25 of SEQ ID NO: 22.

19. The retro-inverso polypeptide of claim 17, wherein the D-amino acids comprise the sequence of SEQ ID NO: 22.

20. A nucleic acid encoding the recombinant polypeptide as defined in any one of claims 1 to 16.

21. A vector comprising the nucleic acid as defined in claim 20.

22. A host cell comprising the nucleic acid as defined in claim 20 or the vector as defined in claim 21.

23. A pharmaceutical composition comprising the recombinant polypeptide as defined in any one of claims 1 to 16, the retro-inverso polypeptide of any one of claims 17 to 19, the nucleic acid as defined in claim 1920, or the vector as defined in claim 21; together with a pharmaceutically acceptable excipient, diluent, or carrier.

24. A method of preventing or reducing interaction of EAG2 and Kvß2 in a cell comprising:
contacting the cell with the recombinant polypeptide as defined in any one of claims 1 to 16, the retro-inverso polypeptide as defined in any one of claims 17 to 19, the nucleic acid as defined in claim 20, the vector as defined in claim 21, or the pharmaceutical composition as defined in claim 23.

25. A use of the recombinant polypeptide as defined in any one of claims 1 to 16, the retro-inverso polypeptide as defined in any one of claims 17 to 19, the nucleic acid as defined in claim 20, the vector as defined in claim 21, or the pharmaceutical composition as defined in claim 23 for preventing or reducing interaction of EAG2 and Kvß2 in a cell.

26. A use of the recombinant polypeptide as defined in any one of claims 1 to 16, the retro-inverso polypeptide as defined in any one of claims 17 to 19, the nucleic acid as defined in claim 20, the vector as defined in claim 21, or the pharmaceutical composition as defined in claim 23 for preparation of a medicament for preventing or reducing interaction of EAG2 and Kvß2 in a cell.

27. The recombinant polypeptide as defined in any one of claims 1 to 16, the retro-inverso polypeptide as defined in any one of claims 17 to 19, the nucleic acid as defined in claim 20, the vector as defined in claim 21, or the pharmaceutical composition as defined in claim 23 for use in preventing or reducing interaction of EAG2 and Kvß2 in a cell.

28. A method of treating cancer in a subject comprising:
administering to the subject the recombinant polypeptide as defined in any one of claims 1 to 16, the retro-inverso polypeptide as defined in any one of claims 17 to 19, the nucleic acid as defined in claim 20, the vector as defined in claim 21, or the pharmaceutical composition as defined in claim 23.

29. A use the recombinant polypeptide as defined in any one of claims 1 to 16, the retro-inverso polypeptide as defined in any one of claims 17 to 19, the nucleic acid as defined in claim 20, the vector as defined in claim 21, or the pharmaceutical composition as defined in claim 23 for treatment of cancer in a subject.

30. A use of the recombinant polypeptide as defined in any one of claims 1 to 16, the retro-inverso polypeptide as defined in any one of claims 17 to 19, the nucleic acid as defined in claim 20, the vector as defined in claim 21, or the pharmaceutical composition as defined in claim 23 for preparation of a medicament for treatment of cancer in a subject.

31. The recombinant polypeptide as defined in any one of claims 1 to 16, the retro-inverso polypeptide of any one as defined in claims 17 to 19, the nucleic acid as defined in claim 20, the vector as defined in claim 21, or the pharmaceutical composition as defined in claim 23 for use in treatment of cancer in a subject.

32. The method of claim 28 or the use of any one of claims 29 to 31, wherein the cancer is a glioma or medulloblastoma.

33. The method or use of claim 32, wherein the cancer is a glioma, preferably a glioblastoma (GBM).

34. The method of claim 33, wherein the GBM is an IDH wild type GBM.

35. The method of claim 33, wherein the GBM is an IDH mutant GBM.

36. The method or use of any one of claim 28 to 35, wherein the cancer is radiation resistant, chemotherapy resistant, targeted therapy-resistant.

37. The method or use any one of claim 28 to 36, wherein the cancer is resistant to temozolomide.

38. The method or use of any one of claims 28 to 37, wherein the cancer is a post-therapy or recurrent.

39. A method of screening for a candidate therapeutic for cancer comprising:
contacting a human cell with a test compound, wherein the cell has an original level of interaction between EAG2 and Kvß2,
measuring a level of interaction between EAG2 between Kvß2 after the step of contacting, and
identifying the test compound as a candidate therapeutic for cancer if the measured level of the interaction between EAG2 and Kvß2 is reduced compared to the original level.

40. The method of claim 39, wherein the cancer is a glioma or medulloblastoma.

41. The method of claim 40, wherein the cancer is a glioma, preferably a glioblastoma (GBM).

42. The method of claim 41, wherein the GBM is an IDH wild type GBM.

43. The method of claim 41, wherein the GBM is an IDH mutant GBM.

44. The method of claim 39, wherein the human cell is from a cell line.

45. The method of any one of claims 39 to 44, wherein the test compound comprises a polypeptide.

46. The method of claim 45, wherein the polypeptide comprises a fragment of human Kvß2 (SEQ ID NO: 26) or EAG2 (SEQ ID NO: 24).

47. The method of any one of claims 39 to 46, wherein the candidate therapeutic is compared to a control comprising the recombinant polypeptide as defined in any one of claims 1 to 16 or the retro-inverso recombinant polypeptide as defined in any one of claims 17 to 19.


SEQUENCE LISTING (26 sequences)

SEQ ID NO: 1 - protein, 25 aa, synthetic construct
YAAGKAEVVLGNIIKKKGWRRSSLV

SEQ ID NO: 2 - protein, 17 aa, synthetic construct
YAAGKAEVVLGNIIKKK

SEQ ID NO: 3 - protein, 19 aa, synthetic construct
KAEVVLGNIIKKKGWRRSS

SEQ ID NO: 4 - protein, 13 aa, synthetic construct
KAEVVLGNIIKKK

SEQ ID NO: 5 - protein, 12 aa, synthetic construct
GRKKRRQRRRPQ

SEQ ID NO: 6 - protein, 14 aa, synthetic construct
CGRKKRRQRRRPQC

SEQ ID NO: 7 - protein, 32 aa, synthetic construct
GDIMGEWGNEIFGAIAGFLGGRKKRRQRRRPQ

SEQ ID NO: 8 - protein, 16 aa, synthetic construct
RQIKIWFQNRRMKWKK

SEQ ID NO: 9 - protein, 18 aa, synthetic construct
CRQIKIWFQNRRMKWKKC

SEQ ID NO: 10 - protein, 27 aa, synthetic construct
GWTLNSAGYLLGKINLKALAALAKKIL

SEQ ID NO: 11 - protein, 29 aa, synthetic construct
CGWTLNSAGYLLGKINLKALAALAKKILC

SEQ ID NO: 12 - protein, 7 aa, synthetic construct
LCLRPVG

SEQ ID NO: 13 - protein, 9 aa, synthetic construct
CLCLRPVGC

SEQ ID NO: 14 - protein, 8 aa, synthetic construct
RRRRRRRR

SEQ ID NO: 15 - protein, 10 aa, synthetic construct
CRRRRRRRRC

SEQ ID NO: 16 - protein, 6 aa, synthetic construct
GSGSGS

SEQ ID NO: 17 - protein, 43 aa, synthetic construct
GRKKRRQRRRPQGSGSGSYAAGKAEVVLGNIIKKKGWRRSSLV

SEQ ID NO: 18 - protein, 45 aa, synthetic construct
CGRKKRRQRRRPQCGSGSGSYAAGKAEVVLGNIIKKKGWRRSSLV

SEQ ID NO: 19 - protein, 35 aa, synthetic construct
GRKKRRQRRRPQGSGSGSYAAGKAEVVLGNIIKKK

SEQ ID NO: 20 - protein, 37 aa, synthetic construct
GRKKRRQRRRPQGSGSGSKAEVVLGNIIKKKGWRRSS

SEQ ID NO: 21 - protein, 21 aa, synthetic construct
RRRRRRRRKAEVVLGNIIKKK

SEQ ID NO: 22 - protein, 43 aa, synthetic construct
VLSSRRWGKKKIINGLVVEAKGAAYSGSGSGQPRRRQRRKKRG

SEQ ID NO: 23 - RNA, 11283 nt, Homo sapiens
  source 1..11283: mol_type = transcribed RNA
gcagttcccgagccccgtgtgcggcaccgccacagtctgggcagcggcggccgggggagcgctactaccatgaactgcctggtcctcctccccagagctgctcatccgggtcgggctggagacacagtcaggggaccccgtcgccgccgccgcgccccctcttctttcggctcaatcttctcttccaccttttcctcctcttcctccaccttctttgcctgcatccccccctcccccgccgcggatcctggccgctgctctccagacccaggatgccggggggcaagagagggctggtggcaccgcagaacacatttttggagaacatcgtcaggcgctccagtgaatcaagtttcttactgggaaatgcccagattgtggattggcctgtagtttatagtaatgacggtttttgtaaactctctggatatcatcgagctgacgtcatgcagaaaagcagcacttgcagttttatgtatggggaattgactgacaagaagaccattgagaaagtcaggcaaacttttgacaactacgaatcaaactgctttgaagttcttctgtacaagaaaaacagaacccctgtttggttttatatgcaaattgcaccaataagaaatgaacatgaaaaggtggtcttgttcctgtgtactttcaaggatattacgttgttcaaacagccaatagaggatgattcaacaaaaggttggacgaaatttgcccgattgacacgggctttgacaaatagccgaagtgttttgcagcagctcacgccaatgaataaaacagaggtggtccataaacattcaagactagctgaagttcttcagctgggatcagatatccttcctcagtataaacaagaagcgccaaagacgccaccacacattattttacattattgtgcttttaaaactacttgggattgggtgattttaattcttaccttctacaccgccattatggttccttataatgtttccttcaaaacaaagcagaacaacatagcctggctggtactggatagtgtggtggacgttatttttctggttgacatcgttttaaattttcacacgactttcgtggggcccggtggagaggtcatttctgaccctaagctcataaggatgaactatctgaaaacttggtttgtgatcgatctgctgtcttgtttaccttatgacatcatcaatgcctttgaaaatgtggatgagggaatcagcagtctcttcagttctttaaaagtggtgcgtctcttacgactgggccgtgtggctaggaaactggaccattacctagaatatggagcagcagtcctcgtgctcctggtgtgtgtgtttggactggtggcccactggctggcctgcatatggtatagcatcggagactacgaggtcattgatgaagtcactaacaccatccaaatagacagttggctctaccagctggctttgagcattgggactccatatcgctacaataccagtgctgggatatgggaaggaggacccagcaaggattcattgtacgtgtcctctctctactttaccatgacaagccttacaaccataggatttggaaacatagctcctaccacagatgtggagaagatgttttcggtggctatgatgatggttggctctcttctttatgcaactatttttggaaatgttacaacaattttccagcaaatgtatgccaacaccaaccgataccatgagatgctgaataatgtacgggacttcctaaaactctatcaggtcccaaaaggccttagtgagcgagtcatggattatattgtctcaacatggtccatgtcaaaaggcattgatacagaaaaggtcctctccatctgtcccaaggacatgagagctgatatctgtgttcatctaaaccggaaggtttttaatgaacatcctgcttttcgattggccagcgatgggtgtctgcgcgccttggcggtagagttccaaaccattcactgtgctcccggggacctcatttaccatgctggagaaagtgtggatgccctctgctttgtggtgtcaggatccttggaagtcatccaggatgatgaggtggtggctattttagggaagggtgatgtatttggagacatcttctggaaggaaaccacccttgcccatgcatgtgcgaacgtccgggcactgacgtactgtgacctacacatcatcaagcgggaagccttgctcaaagtcctggacttttatacagcttttgcaaactccttctcaaggaatctcactcttacttgcaatctgaggaaacggatcatctttcgtaagatcagtgatgtgaagaaagaggaggaggagcgcctccggcagaagaatgaggtgaccctcagcattcccgtggaccacccagtcagaaagctcttccagaagttcaagcagcagaaggagctgcggaatcagggctcaacacagggtgaccctgagaggaaccaactccaggtagagagccgctccttacagaatggagcctccatcaccggaaccagcgtggtgactgtgtcacagattactcccattcagacgtctctggcctatgtgaaaaccagtgaatcccttaagcagaacaaccgtgatgccatggaactcaagcccaacggcggtgctgaccaaaaatgtctcaaagtcaacagcccaataagaatgaagaatggaaatggaaaagggtggctgcgactcaagaataatatgggagcccatgaggagaaaaaggaagactggaataatgtcactaaagctgagtcaatggggctattgtctgaggaccccaagagcagtgattcagagaacagtgtgaccaaaaacccactaagaaaaacagattcttgtgacagtggaattacaaaaagtgaccttcgtttggataaggctggggaggcccgaagtccgctagagcacagtcccatccaggctgatgccaagcaccccttttatcccatccccgagcaggccttacagaccacactgcaggaagtcaaacacgaactcaaagaggacatccagctgctcagctgcagaatgactgccctagaaaagcaggtggcagaaattttaaaaatactgtcggaaaaaagcgtaccccaggcctcatctcccaaatcccaaatgccactccaagtacccccccagataccatgtcaggatatttttagtgtctcaaggcctgaatcacctgaatctgacaaagatgaaatccacttttaatatatatacatatatatttgttaatatattaaaacagtatatacatatgtgtgtatatacagtatatacatatatatattttcacttgctttcaagatgatgaccacacatggattttgatatgtaaatattgcatgtccagctggattctggcctgccaaagaagatgatgattaaaaacatagatattgcttgtatattatgcagttgactgcatgcacactttacatttatttataatctctattctataataaaagagtatgatttttgttacccaaggcaatgtaatatttgaagaatcagggtccaacctgccaatgttgccatgacaaatttgatctcaggctgagtatgtatatcgagctatcattttctcactgttctgttaagttaaaagtatagattaatgccaaggaagagttcagtcttttaaactattagtaccatgttatgatctctttccataggatatttttctgtaacttagtgttaggttcttttcccctacaggcagtgtttgctccatttaaatgactgtttagttgcctaagtcagataagagtgtgggatattgaaaacttattgctatgggttttgtgcatgaattacatttttgatcaactatacctctcgttttttaacattatacttagatagtaatcattagctcttatgtaaggaaatccctactccctccttcttggagaacagctgccatcttgtaagctgacattatcaacatcataaatactaaactattaatatagagtatgttattatagaaacactgcaccacagggtataagggccttttgacaattcatgtaagagatgctgaatttccaaagctgataaatgctctaaatttgtatttccaaaacatgttcaagagtgacattgaaaggcttgttagcaaagaaacatacaaaaaataaagcatttaagtaatcatatcaaaatgctaactattatggtataaaataaaaaagaatgtaaacagatagataaatttttgtggcatgtatataacttagaaagaatgaaacactggtacggattgttgacacatgaatcctagtgtttgtagtgataacacatgaaatgtggtaaaacaaacaaaccaaccaaccaagaagtagacattaagttgctcgactcagataactgcttttctatcattttattgtggttatagttttttaaagctttttgcacacagttctactgctggacttctgctgagttgattcaataagctattgtggctcaggcaggtccatttgtgaaagggggatactaaagactgaattttagtcttttgaattcaaatcctctttgtagaaagtgcctattatcaagtttttaaaaaaagtcacaaagagttgtcctgtatatagctagtcccttaaatgataaggattgagaataaaatacacattcacttgtaagaacccaggagaacttatttcttttgactaaactgtattttaggaagtcgttcagggaagcatacttttcagtcagtgcatgcacagcatttaatatggggagcaggggagttagtgtttgcatgaagttgaaaaaaatgtgtgagtaatttatatattacgggaccatctgtacatatatgttaacaatatgcaaaagatgatggcaaaatcgaaggagatatcttgatctattatacccatttgtatgagcttttctatctcccagtattcccaaccaaagccactagaactgaaagacatgttatcacattcagacttgagaagtgcaaaacagtgatttataaaaacagttgctattttagaatttctattgaacttcacatgcccacaagatgtgcctctttaatctaggagtctcaaaacatatgagccatgggctccattttcctttctttatgcgtataacttccattaaccataatggaagttttcaggggaacacgtctccatgtaatgctacaatatctacctaccacaaacacaagaaaatgaaataattatggagtaaagaatcagccttgaaattggaattttgttgctttcatggtgaggatgatatctttttcgtgacctctgacatttattgaatttcagggtatttttctatttgtttaagtcaagtttggcttaagcttttaaaggtgaatgtgtggttaaaataagaatctgattatagaagttgttagtttaactttagccagtgcttcaaaactaactattggtgtaaagctataaaagaatgactgtgaacaacatgatgatttgaagttctaattcatggctttgcatattagtggtgcaaatatttactgtgttctgacattgacgtagagtttcacctaatcgacctacaatgttattgttcatatatgtgagaaacataatggttttgaattactccagattgttaggaaggagggaaagtatcaattgtttaattatggtgcagttatcatagtgtcttttcagccatctctgttctctgtgtgcatgatatgaagaggtgctgaatgcaccctgctgttttcagcaggaatgcatgttttcagcaaacattgtatatagagtagtcttttaggttcccagattccacaaatcttagcagttgaatttactcagatttgaatgaagggagaatatttgctttaatacatgcaataatgataaaatcatgttctatgttgcctcaactagcacatagacagtagatgctcttttgaggaccagataactgcatgttttattcctacactgtatagatgctcttggtgcatccagaataaaaattttaatattatatttctaaataatatattaataggagtaaatggatatgtattagaaatccaaacattttccaaagcaatttacacaccttcaatatttttctatgatgttattaataggtaagatcagatgctgtatcctcgacaggtggttgtaacaaaatagcaaaagtcaaattagaatggaaatgtgagtgatgtatcataatggcaggttttatcagtctgtatcaggatattgttgcactaaatgtgatctagcagatggaagagattatgataaaaagcaaataggcacacaaaagccagatgacaataccaaagctatatgaatggtactgaaaatggttcaggcacagtggttgaattgaatgacgtggttgaatacaggcccatgcttcattgttaaaatggaaaatttttatgacactagttgcatcttgcattaagtataggactaaaaatgtcctaagtattttttacaaacaactttatgtatatcagagaaaaaaatggaaaatctggggtttgaaattgctctagaaaaaatgtgtacttctgttgtactcaacactttaataggaaaattagttgtgaccgctaactaaagtaccattatcaaaattaaccttattggagcccaaatgaaaatcacattttcccttctgtttaaggagattttgttcaccctgtgatatctggtccatttgatatcttattttaaatagctaaagtcatttggaattttttttaatattagccttcagagcagtgactgttttgaagtgtgcagtggcagatatttgtgaaattattcatcatatttctaatgaattccactatcttgtataatttttatcttttaagcaattttctatgagtttgttttccaaaattccatgtaggtaatgagaacatttatctagaatgtgcatatcatacatggataactttcaaatatgaatattttataatcatgtgaatgttgtgctataattttttctattaggtgtaaaagtatttctcttctcatcttaatggaaagggggaaaagacaaaaacagtcttttggaaaacaccttaagtcttagtatctgagctttgtcttgtgttctgaatttagctttaccacaaactagaagtgtaattatgggcaagttaacctttcctagtgtaccaaatggtaccatttccccgccaataaataagaaaacaaagataacagctgttgcttccttatatcaacatgctattggtcaatgaaaaaattatctttcttgcaggtgaaaatcacattatgacaagtgatcactttttcattctttcatagaagagtatccacgatgtactctctatcaaaatattcttctagattttagaagagatttcagggattgacggtgatgtgatttttatctaaatattcttttaggttttagaagagatttatctggtcagaggttaatggagagaggcatagtttgctagtggcaggtaagcataaaacttcatacttatgtgggaatttagaactacaaatcaaataaacgtggaaagaatgaagctcctttgacagaagagagagagaagagaaagataggtaaattttgtgatttttcttatttctacttaatcatactgcttggcagaaattctcaattttgtgaaaaaattggtaagtggccaaggtatagagtagttcaccaatgtatctatgtaacaaaactgaatgtgtaccccttaaatttatacaaataaaaactagagtagttcatccttcaactttttatattaaaataatcatgacctgttcaagatatatgattccacatgggcagacactcttataacaaaattcttaactatgtgctatgtgttgtaagttttggcctatattctgacttgagattttctatcacttcatttgtaggataagacccctgtgtttgcattctttatgagggtcaggaccactgaggatttggactgggaactagaaagatgatgaaggcaagggttgagaaaagactgattagaatctatctgcttctttactcttaggcttagaagttaaataagttaactaagcttatatttaccaaggtctcactatgataagcattttatttaggttatcaaatttaattcttgcagcaaccccatgaaggtataggttaaggaacttgtccaggatggcatacttgttacatggtaaagttgagtttctaacacaggcagtcagaccttagggagaaacctttattttgtagaatactgacattacaagaactatctagttcaaccatctagtctaggtttttccagacacatacttttaaagatttcttaaaataattcttaaaaattgcactcgataatgtgaatgtaaatgtttggtttttcttgataggtcagagtcaatgacagagtttgaaatcatactgtcttaagcaaaaggtgccctgtcccaacagtggaattttcctttacttctcctaacagttgaaagacatcggccaaagtaaactgtcaaatgtggtatgtttgatatatggtcactggtatcaacattttcttttccagttgaattttaacctaaagaggtaatgcctaatggttaaggaaagcaagagtcattcaggattttttttctataatttctttttctttttaatagtgggatttggatgagactccagaatttatttttaaaatttattttttgactcattgggttttaagtagattattcagatatttttattgattgcactgatgttggcttcttggccaaaccactattaaatacattatccaaatgaactatttctgtttagaatggcaacttgtatccttatttccaaaattaatgagctcacagtatgtatatttacttcagagagtttcaataattttctctaatgaaaaaatgttaattttctgaatgtgatttatttgttctaccctttgagatttctaaagcaagtgtcttgagataaggagaaaaaaacaagatattacaacactacatgctggaatcttcagcccaaaccatcttgttttcacttggctaataactgagccagaggtaatcatagaaaggaagaatcctacttttaaaataatgactgtgaaggaccccactgacaccatccaaagctttttatagtcagctcacttccatcctggcttttacattaccactgcatgattatttaaatgaggtcatgtcttgtactacttgtaaaggcacttggtcaaagaagagtaaaccaaaaagtgcagcttatgaccagagttataactgaggaaaaaggagtgtggagaagggacagcagttgcttccttttaatgagttttctgtatgtggtgccttttaaatagcttttgtttatttgtttttaagatgacacattgtcattaaggttttaacaaaaacctttggaagcagtttgaaagcttgtcaaatgctggggccttctttagaaataagtgttctcccaccaaccaatgagtttagaacctctggttctgacctacaaacccaatcccctaagcacttggctggttgggctgcattttgatgggaggaaacaaaaagatctgatcactctctctcattcatgcggaatcactgactgttgaatgaccaaagcagtcaaagccaaaatattcaccttgaagcttcaataaaccagattgagtatccataataactaatttaagtaattttatccccattctctgctaaatcatatttcataatataaaatgtaaccacttccctctatccaaagcttttggtcagactctccattgagttagagaaattggactttggcaaaaagcaacataaagattattgttgttcctagaaccattgttgccatggacccctcatttgctcctcaactttttcaaccaaattatagcgggaaagattcctgatgaagccaagactgcaaacaggaagaatacctagctagtaaactatgcaaaatcacaacaaagattcatttataattccatgttacagtgtttgatagtcctaagtatttaatattggtatattatgtcacagccatgaactgtcaacttacacaaaatccaaacgcactttcatttaaaatcaagattagcctatttcattttcaggaaaaaacagttgtcagaattgcttttttgacctctgcagggatgttttcacattgctattcttatgaccctttatcagagagggaataagtgtagtgttcactttcatctccttagggttgactgcaccagcatcttccatcaagcagtcgccttccaccttagaacctgttcacagttaataaagcaagggggaatatgcatttcatattaggaacagatttcttaagtgattgttatgaaaagtggaaaccagtttagaaccatttttttaccctaaaacatccattgtagtttgttgaaagagcaatgacttttagaagtgcttggtactcaaacaacacaaaatgtgcagattcacgatatattgaaaattatagagttctttttagtctatgttctagtgactttgtttacactagttgtggcattttgtatactacaatgtaaattctactataacactaatgttactatgggaattaattttgcaacttcttaaatgtttttgtcactgcaaccatattggtgagatattgggggtattaaaaatgcctctgcctttaagggttaattcaccagaaataattgattcatgcattgacaaaggaaacaatgcaaaaatttcatgtgggatgtttttaataccattatgctaagcagtacttgttggcttttttcaaaatctgattcatgctaagcagatccttttttctacttaaacaaagatactgcaatgtataatatagaaattttgtatgtactgttgacctatcacctgactgctgacaggatgtgtgaaaatattttaactttgtgactttagggtcaattatgaaacaattgtgatcaaaattcagttgcttctgggaagtttgtggtgattatcactttatttaacgtttgttctcaaaaaatggtcattttgaaaagtgaattggcaggtggaagcttttagcccaaaagaaatctgggtttcagcacaataaactgtatttcatcagggaggactaactttgctttttttcacactttatttaaatgtggatattttttcatgatacagaataaaatgtgcattttatggaa

SEQ ID NO: 24 - protein, 988 aa, Homo sapiens
MPGGKRGLVAPQNTFLENIVRRSSESSFLLGNAQIVDWPVVYSNDGFCKLSGYHRADVMQKSSTCSFMYGELTDKKTIEKVRQTFDNYESNCFEVLLYKKNRTPVWFYMQIAPIRNEHEKVVLFLCTFKDITLFKQPIEDDSTKGWTKFARLTRALTNSRSVLQQLTPMNKTEVVHKHSRLAEVLQLGSDILPQYKQEAPKTPPHIILHYCAFKTTWDWVILILTFYTAIMVPYNVSFKTKQNNIAWLVLDSVVDVIFLVDIVLNFHTTFVGPGGEVISDPKLIRMNYLKTWFVIDLLSCLPYDIINAFENVDEGISSLFSSLKVVRLLRLGRVARKLDHYLEYGAAVLVLLVCVFGLVAHWLACIWYSIGDYEVIDEVTNTIQIDSWLYQLALSIGTPYRYNTSAGIWEGGPSKDSLYVSSLYFTMTSLTTIGFGNIAPTTDVEKMFSVAMMMVGSLLYATIFGNVTTIFQQMYANTNRYHEMLNNVRDFLKLYQVPKGLSERVMDYIVSTWSMSKGIDTEKVLSICPKDMRADICVHLNRKVFNEHPAFRLASDGCLRALAVEFQTIHCAPGDLIYHAGESVDALCFVVSGSLEVIQDDEVVAILGKGDVFGDIFWKETTLAHACANVRALTYCDLHIIKREALLKVLDFYTAFANSFSRNLTLTCNLRKRIIFRKISDVKKEEEERLRQKNEVTLSIPVDHPVRKLFQKFKQQKELRNQGSTQGDPERNQLQVESRSLQNGASITGTSVVTVSQITPIQTSLAYVKTSESLKQNNRDAMELKPNGGADQKCLKVNSPIRMKNGNGKGWLRLKNNMGAHEEKKEDWNNVTKAESMGLLSEDPKSSDSENSVTKNPLRKTDSCDSGITKSDLRLDKAGEARSPLEHSPIQADAKHPFYPIPEQALQTTLQEVKHELKEDIQLLSCRMTALEKQVAEILKILSEKSVPQASSPKSQMPLQVPPQIPCQDIFSVSRPESPESDKDEIHF

SEQ ID NO: 25 - RNA, 3869 nt, Homo sapiens
  source 1..3869: mol_type = transcribed RNA
gtccccagcgccatgggctgtgctacctgcagtaacgcagactgcgagccccggctgcctctccagtgctctctctcctctggccttcagcaggctgggtcacagagaattttcccactgtaaaaaaccgagcaagtctggataagtgaggctggctccatgtatccagaatcaacgacgggctccccggctcggctctcgctgcggcagacgggctcccccgggatgatctacagtactcggtatgggagtcccaaaagacagctccagttttacaggaacctgggcaagtctggcctgcgggtctcctgcctgggacttggaacatgggtgaccttcggaggccagatcaccgatgagatggcagagcagctcatgaccttggcctatgataatggcatcaacctcttcgatacagcagaagtctacgcagccggcaaggctgaagtggtactgggaaacatcattaagaagaaaggatggaggcggtccagcctcgtcatcaccaccaagatcttctggggcggaaaggcggagacggagcggggcctgtccaggaagcacataatcgaaggtctgaaagcttccctggagcgactgcagctggagtacgtggatgtggtgtttgccaaccgcccggaccccaacaccccgatggaagagaccgtccgcgccatgacccacgtcatcaaccaggggatggccatgtactggggcacgtcacgctggagctccatggagatcatggaggcctactccgtggcccggcagttcaacctgaccccgcccatctgcgagcaggctgagtaccacatgttccagcgtgagaaagtggaggtgcagctgccggagctgttccacaagataggagtgggcgccatgacctggtcccctctggcctgtggcattgtttctggcaagtacgacagtggcatcccaccctactcaagagcctccttgaagggctaccagtggctgaaggacaagatcctcagtgaggagggccggcgccagcaagccaagctgaaggagctgcaggccatcgccgagcgcctgggctgcaccctgccccagctggccatagcctggtgcctgaggaatgagggagtcagctccgtgctcctgggggcctccaatgcggaccagctcatggagaacattggggcaatacaggtccttccgaaactgtcatcttccattatccacgagattgatagtattttgggcaataaaccctacagcaaaaaggactacagatcctaagccgcccccgcccgcctgctcggacagtttccgttccctcctagtctctgttcgctcgcttaagctgttttgaagccaagtgaagagtgtggtttgcatccaagagaaaacaccacactgtgatgtcatcgggaaatgatctcccaagtcgctgccagacaccacccactgcttcgccggacaatgtcgaagtccagtctgtgccggggaaggcactggttaggaaggatgttcaaacggtcccacccaagcctgtcacctctgctcatcctccaagaccacccagctttctcccagccacagccaagattcccaaagtcaaggcccaaagatttccaaggttcccaaagtcaaggccaggccaaggcctggttgggtccttggggcgggcagggccagcctctcctctgctgagaatccccacttggtgtagggggagaggggaaaggggtctggcccatcgaggggccccttctgccagggccttggttgctggggcagggcctccccactgggggtcttcctccacctcccactttccaagggctccaggaatctggggcctgaccacagattcctctcccatccttttctgctccaacctgccccactgggtcccggcaggggccatgcctaccaagctcgagctggcccttgacccccacccacccccacccttgctggcaggggcagggaccccagggggattgactctgcagtttgggagccacaaaaagcgtagcggtgtgatttctagctcagcctcccaccgtcttcctcctacacaccaatgatgagcctcatgccagtgaggcccggagcgcttgggaggggtcccagtggggcaggcccctctgtctggccacccctctgtcctggccccggaaggccctgtggtcatgtgctcctagctgcacggtggctgctggccacaccacggcaagtggcagcaggggccggccctgtgcacaaggatgcactcctctcggcccctgtagactttctctaaagccgcccgccagcccaggccgctgctctgcaccgagctggtgggcttgggttttgtggagcgcatgcttggaccctttcagtaaggaagggtctttggggttttctgtgcccatgacttgggggctgcacccccacagcacccccacaatgtaggaaaagacctcagggaacctctccctggaaagacgggcagggctggttagcccctcccactgcctgacacctgggacaggctgggcagaggggagagagggcaggacaggccagagtgacgcccccgtgcagcttgggccggagggcaagggatgccagtaagtctgcaggtgcggggtgccacctacaggcccaggcctgtgtcccaagcagtacccaggctttgcagaccacgcggggcagggctccactgaagccacccccacccctcgccagctagctccatagggaagcctgtgtctcctgcccccagggcgcaccctcagtgcaggcacctctgttcccgctttgcccctggaggagccactattccagaaggctccaccctgccgtcctgcgggagcctgctgtccagtcctggccgggccaaggcctgggaaactgtgaaagtcagaaaggccagcggggagaggctggggcgaggggaggagggggatcagcttctgctattaccgaccccccttcatgctgcccctggcgcctagaacccttgcccctcctcatagaccaagtcccgggggtctccactcagtcctgctgcctgcttcaccagaagcagccctgtgagtgtggggtggggaagtcccttcccaacggaggtcccagcctatggccctgggcccaggtgggggtcgcctgcttccttcccggacagggtcctgcagtggccaatggtgccagagggcaggtggcccaccctcgccatcagggagggtggctggccccatccccactgccacccagccccacccactgttggaagagggaccagcgcgaggtggtgcccagggtgggcactgctgcttaatgcgaggcacacctggggcagctgagcccccaaaggctgcgggtttgccaaacacagagaggccaggccccagtgtcaggatgcagtcagcctctggcgcagctctttccacgacctggttcctggatgtcctgcttgctccacacccatctacagggaggatgtgagggggctctgcctcctagggccaggtcccccctctcgggaggaggtattgggtaggaccatccaagaaagggcagaagaccaagggcagtcggggtctagaaaggagggcgctggccctgctggacgcttcggagcccccactgtttcccactcagctttgtgctcagatcccaggtcccaaggagtgacaggggcttcctcccaccttctgtccttgtccagtcatgtaaataatgtgctttttctctccccgagtctttttttttaaacctaccgtggttcctcagctaactgcattccctacccaggcagagactgtcctatgcctcgagcttccaaacgagactcagaccgcgacacagccaccgtatttatggaatgacaaaataaataaagcccaaaccca

SEQ ID NO: 26 - protein, 367 aa, Homo sapiens
MYPESTTGSPARLSLRQTGSPGMIYSTRYGSPKRQLQFYRNLGKSGLRVSCLGLGTWVTFGGQITDEMAEQLMTLAYDNGINLFDTAEVYAAGKAEVVLGNIIKKKGWRRSSLVITTKIFWGGKAETERGLSRKHIIEGLKASLERLQLEYVDVVFANRPDPNTPMEETVRAMTHVINQGMAMYWGTSRWSSMEIMEAYSVARQFNLTPPICEQAEYHMFQREKVEVQLPELFHKIGVGAMTWSPLACGIVSGKYDSGIPPYSRASLKGYQWLKDKILSEEGRRQQAKLKELQAIAERLGCTLPQLAIAWCLRNEGVSSVLLGASNADQLMENIGAIQVLPKLSSSIIHEIDSILGNKPYSKKDYRS